SEQUENCE LISTING (21 sequences)

SEQ ID NO: 1 - DNA, 20 bp, Artificial Sequence
  Forward primer for human cytochrome B
atgaccccaatacgcaaaat

SEQ ID NO: 2 - DNA, 20 bp, Artificial Sequence
  Reverse primer for human cytochrome B
cgaagtttcatcatgcggag

SEQ ID NO: 3 - DNA, 20 bp, Artificial Sequence
  Forward primer for human cytochrome C oxidase subunit III
atgaccccaatacgcaaaat

SEQ ID NO: 4 - DNA, 20 bp, Artificial Sequence
  Reverse primer for human cytochrome C oxidase subunit III
cgaagtttcatcatgcggag

SEQ ID NO: 5 - DNA, 20 bp, Artificial Sequence
  Forward primer for human NADH dehydrogenase
atacccatggccaacctcct

SEQ ID NO: 6 - DNA, 20 bp, Artificial Sequence
  Reverse primer for human NADH dehydrogenase
gggcctttgcgtagttgtat

SEQ ID NO: 7 - DNA, 20 bp, Artificial Sequence
  Forward primer for rat cytochrome B
tccacttcatcctcccattc

SEQ ID NO: 8 - DNA, 20 bp, Artificial Sequence
  Reverse primer for rat cytochrome B
ctgcgtcggagtttaatcct

SEQ ID NO: 9 - DNA, 19 bp, Artificial Sequence
  Forward primer for rat cytochrome C oxidase subunit III
acataccaaggccaccaac

SEQ ID NO: 10 - DNA, 20 bp, Artificial Sequence
  Reverse primer for rat cytochrome C oxidase subunit III
cagaaaaatccggcaaagaa

SEQ ID NO: 11 - DNA, 20 bp, Artificial Sequence
  Forward primer for rat NADH dehydrogenase
caataccccacccccttatc

SEQ ID NO: 12 - DNA, 20 bp, Artificial Sequence
  Reverse primer for rat NADH dehydrogenase
gaggctcatcccgatcatag

SEQ ID NO: 13 - DNA, 20 bp, Artificial Sequence
  Forward primer for bacterial 16S ribosomal RNA
cgtcagctcgtgttgtgaaa

SEQ ID NO: 14 - DNA, 19 bp, Artificial Sequence
  Reverse primer for bacterial 16S ribosomal RNA
ggcagtctccttgagttcc

SEQ ID NO: 15 - DNA, 20 bp, Artificial Sequence
  Forward primer for human cytochrome C oxidase subunit III
atgacccaccaatcacatgc

SEQ ID NO: 16 - DNA, 20 bp, Artificial Sequence
  Reverse primer for human cytochrome C oxidase subunit III
atcacatggctaggccggag

SEQ ID NO: 17 - DNA, 20 bp, Artificial Sequence
  Forward primer for human GAPDH
agggccctgacaactctttt

SEQ ID NO: 18 - DNA, 20 bp, Artificial Sequence
  Reverse primer for human GAPDH
ttactccttggaggccatgt

SEQ ID NO: 19 - DNA, 20 bp, Artificial Sequence
  Forward primer for rat GAPDH
gaaatcccctggagctctgt

SEQ ID NO: 20 - DNA, 20 bp, Artificial Sequence
  Reverse primer for rat GAPDH
ctggcaccagatgaaatgtg

SEQ ID NO: 21 - DNA, 16571 bp, Homo sapiens
gatcacaggtctatcaccctattaaccactcacgggagctctccatgcatttggtattttcgtctggggggtgtgcacgcgatagcattgcgagacgctggagccggagcaccctatgtcgcagtatctgtctttgattcctgcctcattctattatttatcgcacctacgttcaatattacaggcgaacatacctactaaagtgtgttaattaattaatgcttgtaggacataataataacaattgaatgtctgcacagccgctttccacacagacatcataacaaaaaatttccaccaaacccccccctccccccgcttctggccacagcacttaaacacatctctgccaaaccccaaaaacaaagaaccctaacaccagcctaaccagatttcaaattttatctttaggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaagggacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactgagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagatcaaaacactgaactgacaattaacagcccaatatctacaatcaaccaacaagtcattattaccctcactgtcaacccaacacaggcatgctcataaggaaaggttaaaaaaagtaaaaggaactcggcaaaccttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcatgacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatggctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgcaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgatagccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatggtggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaactaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgagccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaagcacataatttgaatcaacacaaccacccacagcctaattattagcatcatccccctactattttttaaccaaatcaacaacaacctatttagctgttccccaaccttttcctccgaccccctaacaacccccctcctaatactaactacctgactcctacccctcacaatcatggcaagccaacgccacttatccagcgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacggactcacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcattttcctaattaccatactaatcttagttaccgctaacaacctattccaactgttcatcggctgagagggcgtaggaattatatccttcttgctcatcagttgatgatacgcccgagcagatgccaacacagcagccattcaagcagtcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaaccactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcagtactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaattaaccccctaataaaattaattaaccactcattcatcgacctccccaccccatccaacatctccgcatgatgaaacttcggctcactccttggcgcctgcctgatcctccaaatcaccacaggactattcctagccatacactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttacccttcattattgcagccctagcagcactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagacgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaacccacatcaaaccccccccccccatgcttacaagcaagtacagcaatcaaccttcaactatcacacatcaactgcaactccaaagccacccctcacccactaggataccaacaaacctacccacccttaacagtacatagtacataaagtcatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggccataaagcctaaatagcccacacgttccccttaaataagacatcacgatg

(19) United States
(12) Patent Application Publication
(10) Pub. No.: US 2012/0263736 A1
(43) Pub. Date: Oct. 18, 2012

(54) METHOD FOR PREDICTING AND TREATING A STERILE INFLAMMATION AND DISCRIMINATING BETWEEN STERILE AND INFECTIVE INFLAMMATION

(75) Inventor: Carl J. Hauser, Boston, MA (US)

(73) Assignee: Beth Israel Deaconess Medical Center, Inc., Boston, MA (US)

(21) Appl. No.: 13/513,137

(22) PCT Filed: Dec. 3, 2010
(86) PCT No.: PCT/US10/58877

§ 371 Date: Jul. 5, 2012

Related U.S. Application Data
(60) Provisional application No. 61/266,990, filed on Dec. 4, 2009.

(51) Int. Cl. C12Q 1/68; A61K 31/4706; A61P 31/00; A61K 31/7088; A61P 29/00; A61K 38/13; A61K 39/395

(52) U.S. Cl. 424/172.1; 435/6.15; 514/20.5; 514/313; 514/44 R; 435/6.12

(57) ABSTRACT

The invention provides methods for predicting the likelihood that a subject will develop a sterile inflammation or will have an increased propensity to later develop a sterile inflammation, for determining whether a subject with tissue damage should be administered an antimicrobial agent (at full or a reduced dosage) or an anti-inflammatory agent, and for treating a subject with tissue damage. In the methods, an increased ratio of the amount of mitochondrial nucleic acid or peptide to the amount of microbial nucleic acid or peptide indicates a subject that has an increased likelihood of developing a sterile inflammation or an increased propensity to later develop a sterile inflammation, or a subject that should not be administered, or that should be administered at a reduced dosage, an antimicrobial agent or one or more anti-inflammatory agents and not an antimicrobial agent. Kits for detecting mitochondrial and/or microbial nucleic acids or peptides are provided.

### BACKGROUND OF THE INVENTION

The present invention relates to the clinical diagnosis or prediction of the future development of a sterile inflammation and methods of treating a sterile inflammation.

Injury (e.g., tissue damage resulting from blunt trauma, surgery, hypotension, hypofusion/reperfusion injury, pancreatitis, or shock) causes a systemic inflammatory response syndrome (SIRS) that presents, clinically, much like sepsis. Microbial pathogen-associated molecular patterns (PAMPs) activate innate immunocytes through pattern recognition receptors. Similarly, tissue trauma can release endogenous damage-associated molecular patterns (DAMPs) that activate immunity. Because the inflammatory pathways activated by PAMPs and DAMPs are identical, “sterile” SIRS caused by tissue trauma is currently difficult to distinguish from “infective” SIRS caused by bacterial infection.

There is currently no clinically accepted test to identify patients who have sterile rather than infective SIRS nor are there methods for identifying whether a subject has an increased likelihood of later developing a sterile inflammation. In current clinical practice, a patient diagnosed with SIRS is treated with antibiotics, often with significant potential negative side effects, while a blood culture is done to test for sepsis: a procedure that typically takes 72 hours. Even if sepsis is not found, it is often still assumed that sepsis is present because of the lack of an affirmative test for sterile SIRS. Thus, the lack of an ability to discriminate between sterile and infective SIRS causes current treatment regimes to be unnecessarily hazardous to patients and wasteful. Accordingly, there is a need to develop a rapid test that accurately discriminates between sterile and infective SIRS, so that patients may receive appropriate treatment at the early stages of the inflammatory event. There is also a need to identify subjects (e.g., subjects that do not demonstrate any symptoms of systemic inflammation) at increased risk of later developing a sterile inflammation. Such identification of subjects at risk of developing an infective inflammation (e.g., a sterile systemic inflammation) may be administered anti-inflammatory agents in order to reduce the severity or decrease the propensity to later develop a sterile inflammation (e.g., a sterile systemic inflammation).

### SUMMARY OF THE INVENTION

The present invention stems from our discovery that injury mediates the early release of mitochondrial DAMPs (MTD) into the circulation. This has important functional consequences. MTD contain both formyl peptides and mitochondrial DNA that activate human neutrophils (PMN) through formyl peptide receptor-1 and TLR9, respectively. MTD promote PMN Ca²⁺ flux and MAP Kinase phosphorylation, causing PMN migration and degranulation in vitro and in vivo. Circulation of MTD causes neutrophilic organ injury. Mitochondrial DAMPs cause inflammation because of evolutionary similarity to bacterial PAMPs, and link trauma to inflammation and SIRS. The discovery of mtDNA and mitochondrial peptides as mediators of sterile systemic inflammation allows for the development of methods of treating patients with tissue damage, as well as assays to predict the likelihood that a subject will develop a sterile inflammation, to identify subjects early in their illness with an increased propensity to later develop a sterile inflammation, and to determine whether a subject with tissue damage should be administered an antimicrobial agent or a reduced dosage of an antimicrobial agent.

Accordingly, the invention provides methods of predicting the likelihood that a subject will develop a sterile inflammation requiring the steps of: (a) measuring the amount of microbial (e.g., bacterial) nucleic acid or peptide in a sample from the subject; (b) measuring the amount of a mitochondrial nucleic acid or peptide in the sample; and (c) determining whether the subject has an increased likelihood of later developing a sterile inflammation by comparing the amount of microbial (e.g., bacterial) nucleic acid or peptide measured in step (a) with the amount of mitochondrial nucleic acid or peptide in step (b), where an increased ratio of the amount of mitochondrial nucleic acid or peptide to the amount of microbial (e.g., bacterial) nucleic acid or peptide indicates a subject with an increased likelihood of later developing a sterile inflammation.

The invention also provides methods of identifying a subject with an increased propensity to develop a sterile inflammation requiring the steps of: (a) measuring the amount of microbial (e.g., bacterial) nucleic acid or peptide in a sample from the subject; (b) measuring the amount of a mitochondrial nucleic acid or peptide in the sample; and (c) comparing the amount of microbial (e.g., bacterial) nucleic acid or peptide measured in step (a) to the amount of mitochondrial nucleic acid or peptide measured in step (b), where an increased ratio of the amount of mitochondrial nucleic acid or peptide to the amount of microbial (e.g., bacterial) nucleic acid or peptide identifies a subject as having an increased propensity to later develop a sterile inflammation.

The invention further provides methods of determining whether a subject with tissue damage should be administered an antimicrobial agent or a reduced dosage of an antimicrobial agent, requiring the steps of: (a) measuring the amount of microbial (e.g., bacterial) nucleic acid or peptide in a sample from the subject; (b) measuring the amount of mitochondrial nucleic acid or peptide in the sample; and (c) comparing the amount of microbial (e.g., bacterial) nucleic acid or peptide measured in step (a) with the amount of mitochondrial nucleic acid or peptide measured in step (b), wherein a subject having an increased ratio of the amount of mitochondrial nucleic acid or peptide to the amount of microbial (e.g., bacterial) nucleic acid or peptide should not be administered an antimicrobial agent or administered a reduced dosage of an antimicrobial (e.g., antibacterial) agent.

In another aspect, the invention provides methods of treating a subject with tissue damage, requiring the steps of: (a) measuring the amount of microbial (e.g., bacterial) nucleic acid or peptide in a sample from the subject; (b) measuring the amount of a mitochondrial nucleic acid or peptide in the sample; and (c) comparing the amount of microbial (e.g., bacterial) nucleic acid or peptide measured in step (a) with the amount of mitochondrial nucleic acid or peptide measured in step (b); and (d) administering to the subject having an increased ratio of the amount of mitochondrial nucleic acid or peptide to the amount of microbial (e.g., bacteria]) nucleic acid or peptide one or more anti-inflammatory agents and not administering, or administering at a reduced dosage, an antimicrobial (e.g., antimicrobial) agent.

The invention also provides methods of determining whether a systemic inflammation in a subject is an infective inflammation, a sterile inflammation, or both, that require the steps of: (a) measuring the amount of a microbial (e.g., bacterial) nucleic acid or peptide in a sample derived from the subject; (b) measuring the amount of a mitochondrial nucleic acid or peptide in the sample; and (c) determining whether the systemic inflammation is an infective inflammation, a sterile inflammation, or both based on the measured amounts of microbial (e.g., bacterial) nucleic acid or peptide and mitochondrial nucleic acid or peptide.

In any of the above methods, the subject may have experienced tissue damage (e.g., tissue damage that occurs as a result of surgery, hypotension, hypofusion/reperfusion injury, chemotherapy, pancreatitis, or shock). In a further aspect of all the above methods, the tissue damage may not be a result of blunt trauma.

In additional aspects of all the above methods, the sample may be obtained from the subject within 2 hours of tissue damage (e.g., within 1 hour, within 30 minutes, or within 15 minutes of tissue damage). In any of the above methods, the subject may not demonstrate any significant symptoms of systemic inflammation. In additional embodiments of the above methods, the sterile inflammation is a systemic inflammation (a sterile systemic inflammation).

In further embodiments of the above methods, the mitochondrial nucleic acid or peptide may be absent in bacteria. In any of the above methods, the mitochondrial nucleic acid may encode cytochrome B, cytochrome C oxidase subunit III, or NADH dehydrogenase. In desirable embodiments, the mitochondrial nucleic acid encodes cytochrome B. In other desirable embodiments, the mitochondrial peptide is a formyl peptide.

In additional embodiments of the above methods, the microbial (e.g., bacterial) nucleic acid or peptide is absent in (not endogenous to) humans. In a preferred embodiment, the microbial (e.g., bacterial) nucleic acid is 16S ribosomal DNA or 16S ribosomal RNA. In other preferred embodiments, the microbial (e.g., bacterial) peptide is a formyl peptide.

In any of the above methods, the determining step includes calculating a ratio of mitochondrial nucleic acid to microbial (e.g., bacterial) nucleic acid. In additional embodiments of the above methods, the ratio and amounts of nucleic acid or peptide are converted into a confidence interval (e.g., a confidence interval indicating that the systemic inflammation is infective, sterile, or both).

In any of the methods of the present invention, the amounts of microbial (e.g., bacterial) nucleic and mitochondrial nucleic acid may be measured, for example, by polymerase chain reaction (PCR). Amounts of microbial (e.g., bacterial) peptide and mitochondrial peptide may be measured, for example, by mass spectrometry. Other means of nucleic acid and protein detection include microarrays and multiple analyte protein detection systems.

Additional embodiments of the above methods further include the step of treating the subject with one or more (e.g., two, three, or four) antimicrobial (e.g., antibacterial) agents if the systemic inflammation has been determined to be an infective inflammation.

The invention also provides kits. The provided kits include (a) one or more first oligonucleotide primers effective for the amplification of a microbial (e.g., bacterial) nucleic acid; and (b) one or more second oligonucleotide primers effective for the amplification of a mitochondrial nucleic acid. The provided kits further include instructions for using said first and second oligonucleotide primers to: determine whether a systemic inflammation in a subject is an infective inflammation, a sterile inflammation, or both; determine the likelihood that a subject will develop a sterile inflammation; identify a subject with an increased propensity to later develop a sterile inflammation; or determine whether a subject with tissue damage should be administered an antimicrobial (e.g., antibacterial) agent or a reduced dosage of an antimicrobial agent.

In additional embodiments of the above kits, the microbial (e.g., bacterial) nucleic acid is absent in humans. In a preferred embodiment, the microbial (e.g., bacterial) nucleic acid is 16S ribosomal DNA or 16S ribosomal RNA. In a more preferred embodiment, the kit includes oligonucleotide primers having the sequences 5′-cgtcagctcgtgttgtgaaa-3′ (SEQ ID NO: 13) and 5′-ggcagtctccttgagttcc-3′ (SEQ ID NO: 14).

In further embodiments of the above kits, the mitochondrial nucleic acid is absent in bacteria. In desirable embodiments of the above kits, the mitochondrial nucleic acid encodes cytochrome B, cytochrome C oxidase subunit III, or NADH dehydrogenase. In more desirable embodiments, the mitochondrial nucleic acid encodes cytochrome B. Additional embodiments of the above kits include oligonucleotide primers for human cytochrome B, such as 5′-atgaccccaatacgcaaaat-3′ (SEQ ID NO: 1) and 5′-cgaagtttcatcatgcggag-3′ (SEQ ID NO: 2).

In any of the methods or kits of the present invention, the sterile inflammation may be a systemic inflammation (e.g., a sterile systemic inflammation) or the systemic inflammation may be systemic inflammatory response syndrome (SIRS).

By “anti-inflammatory agent” is meant an agent that reduces (e.g., by at least 10%, 15%, 20%, 25%, 30%, 35%, 40%, 45%, 50%, 55%, 60%, 65%, 70%, 75%, 80%, 85%, 90%, or 95%) one or more (e.g., two, three, four, or five) symptoms of inflammation when administered (e.g., orally, intravenously, intraarterially, and subcutaneously) to a subject. Non-limiting examples of anti-inflammatory agents include non-steroidal anti-inflammatory agents (e.g., ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lomoxicam, isoxicam, mefenaic acid, meclofenamic acid, tolfenamic acid, celecoxib, rofecoxib, valdecoxib, parecoxib, lumiracoxib, etoricoxib, firoxocib, nimesulide, and licofelone), immunosuppressive agents (e.g., methotrexate, azathioprine, basiliximab, daclizumab, cyclosporine, tacrolimus, sirolimus, voclosporin, infliximab, etanercept, adalimumab, mycophenolic acid, fingolimod, pimecrolimus, thalidomide, lenalidomide, anakinra, deferolimus, everolimus, temsirolimus, zotarolimus, biolimus A9, and elsilimomab), corticosteroids (e.g., hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate), cyclosporine H, anti-FPR antibodies, chloroquin, CpG oligodeoxynucleotides (e.g., CpG oligodeoxynucleotides containing modified nucleotide monomers, such as LNA), and anti-TLR9 antibodies.

By “antimicrobial agent” is meant an agent that kills or inhibits (e.g., by at least 10%, 20%, 30%, 40%, 50%, 60%, 70%, 80%, 90%, or 100%) the growth of a microorganism (e.g., a bacterium, fungus, or protozoa) when administered to a subject. Non-limiting examples of antimicrobial agents include: amikacin, gentamycin, kanamycin, neomycin, netilmicin, tobramycin, paromomycin, geldanamycin, herbimycin, loracarbef, ertapenem, doripenem, imipenem, meropenem, cefadroxil, cefazolin, cefalotin, cefalexin, cefaclor, cefamandole, cefoxitin, cefprozil, cefuroxime, cefixime, cefdinir, cefditoren, cefotaxime, cefpodoxime, ceftazidime, ceftibuten, ceftizoxime, ceftriaxone, cefepime, ceftobiprole, teicoplanin, vancomycin, telavancin, clindamycin, lincomycin, azithromycin, clarithromycin, dirithromycin, erythromycin, roxithromycin, troleandomycin, telithromycin, telithromycin, spectinomycin, aztronam, furazolidone, nitrofurantoin, nitrofurantoin, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, bacitracin, colistin, polymyxin B, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfonamidochrysoidine, sulfacetamide, sulfadiazine, silver sulfadiazine, sulfamethizole, sulfamethoxazole, sulfanilamide, sulfasalazine, sulfisoxazole, trimethoprim, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, linezolid, metronidazole, mupirocin, platensimycin, quinupristin, rifaximin, thiamphenicol, and tinidazole.

By “amount” is meant either a mass or a molar quantity of a nucleic acid or peptide.

When a nucleic acid or peptide is herein referred to as being “absent” in an organism (e.g., human or bacteria), it is meant that the nucleic acid or peptide is not identically present in the genome of the organism as indicated by bioinformatics tools (e.g., BLAST or FASTA) for sequence comparison.

As used herein, “amplify” is meant the in vitro amplification of a nucleic acid of interest using, e.g., PCR and real-time PCR.

By the term “blunt trauma” is meant the non-pathologic application of an external force (e.g., by accidental injury or physical attack) on a body part of a subject. Non-limiting examples of blunt trauma include concussions, crushing, abrasions, and lacerations. Non-limiting causes of blunt trauma include motor vehicle/motorcycle crashes and falling.

By “chemotherapy” is meant a therapeutic treatment for a cancer that selectively kills or decreases (e.g., by at least 10%, 20%, 30%, 40%, 50%, 60%, 70%, 80%, or 90%) the proliferation of cancer cells relative to healthy non-cancerous cells in a subject.

The term “effective,” used in the context of diagnostic assays, indicates that an oligonucleotide primer can be used, under a certain set of amplification conditions (e.g., pH, temperature, reaction time, number of amplification cycles, and buffer concentrations) to amplify a nucleic acid of interest.

By “hypotension” is meant a decreased in blood pressure (e.g., a systolic blood pressure of less than 90 mm Hg and/or a diastolic blood pressure of less than 60 mm Hg, or a systolic blood pressure of less than 80 mm Hg and/or diastolic blood pressure of less than 50 mm Hg). Hypotension may also be associated with one of more of the following symptoms: chest pain, shortness of breath, irregular heartbeat, loss of consciousness, and/or seizures.

By “hypofusion/reperfusion injury” is meant damage to tissue that results when an oxygenated blood supply returns to a tissue after a period of ischemia (e.g., period of ischemia greater than 1 minute, 5 minutes, 10 minutes, 30 minutes, 1 hour, 2 hours, 3 hours, 4 hours, 5 hours, or 6 hours). The decrease in oxygen and nutrients provided by blood to a tissue creates a state, in which the restoration of blood to the tissue causes damage through induction of oxidative stress. Hypofusion/reperfusion injury may be caused, for example, by surgery or cardiomyopathy. Hypofusion/reperfusion injury is also commonly referred to as ischemic/reperfusion injury.

By “increased propensity to develop a sterile inflammation” is meant a subject that has at least a 10% (e.g., at least 15%, 20%, 25%, 30%, 35%, 40%, 45%, 50%, 55%, 60%, 65%, 70%, 75%, 80%, 85%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, 500%, 550%, 600%, 650%, 700%, 750%, 800%, 850%, 900%, 950%, 1000%, 1050%, 1100%, 1150%, 1200%, 1250%, 1300%, 1350%, 1400%, 1450%, 1500%, 1550%, 1600%, 1650%, 1700%, 1750%, 1800%, 1850%, 1900%, 1950%, 2000%, 2500%, 3000%, 3500%, 4000%, 4500%, or 5000%) increased risk of later developing (e.g., at least 3 hours, 6 hours, 9 hours, 12 hours, 15 hours, 18 hours, 21 hours, 24 hours, 27 hours, 30 hours, 36 hours, 39 hours, 42 hours, 45 hours, 48 hours, or 3 days) a sterile inflammation (e.g., a sterile systemic inflammation).

By “measure,” in the context of measuring the amount of a nucleic acid in a sample, is meant quantitating a mass or a molar amount of the nucleic acid. Ways of measuring nucleic acids are well known in the art and include, e.g., quantitative polymerase chain reaction (real-time qPCR). Non-limiting methods for measuring mitochondrial nucleic acid and microbial (e.g., bacterial) nucleic acid are described herein. The term measure may also be used in the context of measuring the amount of a peptide or polypeptide in a sample. Non-limiting methods for measuring mitochondrial peptides and bacterial peptides are also described herein.

By “oligonucleotide primer” is meant an oligonucleotide, typically synthetic, that is useful for specifically binding and amplifying a sequence of interest by primer extension.

By “pancreatitis” is meant the inflammation of the pancreas. The term pancreatitis may refer to acute or chronic pancreatitis. Non-limiting examples of symptoms of pancreatitis include severe abdominal pain, nausea, vomiting, increased heart rate, and increased respiratory rate.

By “ratio” is meant either a mass ratio or a molar ratio of nucleic acids or proteins. For a raw ratio obtained from measured nucleic acid, amounts may be normalized in various ways (e.g., for relative nucleic acid lengths, amplification biases, and other experimental considerations), before it is assessed against a cutoff value, used to determine a confidence level, or used to calculate the ratio. For a raw ratio obtained from measured proteins, amounts may be normalized to a control protein (e.g., the expression level of a house-keeping protein, such as β-actin), before it is assessed against a cutoff value, used to determine a confidence level, or used to calculate the ratio. Non-limiting examples of ratios of the amount of mitochondrial nucleic acid to the amount of microbial (e.g., bacterial) nucleic acid include a ratio of at least 25:1, 50:1, 75:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, 1600:1, 1700:1, 1800:1, 1900:1, or 2000:1. Non-limiting examples of ratios of the amount of mitochondrial peptides to the amount of microbial (e.g., bacterial) peptides include a ratio of at least 5.0:1, 6.0:1, 7.0:1, 8.0:1, 9.0:1, 10:1, 11:1, 12:1, 13:1, 14:1, 15:1, 16:1, 17:1, 18:1, 19:1, 20:1, 25:1, 26:1, 27:1, 28:1, 29:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, or 200:1

By “sample” is meant any specimen (e.g., blood, serum, plasma, urine, saliva, amniotic fluid, cerebrospinal fluid, tissue (e.g., placental or dermal), pancreatic fluid, chorionic villus sample, and cells) taken from a subject. Preferably, the sample is taken from a portion of the body affected by sterile inflammation (e.g., a sterile systemic inflammation).

By “shock” is meant an inadequate perfusion of blood to a tissue in a subject. Non-limiting symptoms of shock include tachycardia, hypotension, hypoxemia, and tachypnoea.

By “subject” is meant any animal (e.g., human, cat, dog, horse, monkey, mouse, rat, and rabbit).

By “symptoms of systemic inflammation” is meant one or more (e.g., two, three, or four,) physical manifestations of a systemic inflammatory response (e.g., a sterile inflammation or a infective inflammation). Non-limiting examples of symptoms of systemic inflammation include: altered body temperature (e.g., less than 36° C. or greater than 38° C.), increased heart rate (e.g., greater than 90 beats per minute), tachypnea (e.g., greater than 20 breaths per minute), decreased arterial pressure of CO₂ (e.g., less than 4.3 kPa), altered white blood count (e.g., less than 4,000 cells/mm³ or greater than 12,000 cells/mm³), increased histamine levels (e.g., greater than 60 ng/mL in blood), increased leukotriene B4 levels (e.g., greater than 30 pg/mL or greater than 35 pg/mL in blood), increased prostaglandin levels (e.g., greater than 3.0 ng/mL in blood), increased levels of pro-inflammatory cytokines (e.g., greater than 20 ng/mL TNF-α and/or greater than 10 pg/mL IL-6).

By the term “systemic inflammation” refers to any immune-mediated inflammatory state affecting multiple portions of the body. One type of systemic inflammation is systemic inflammatory response syndrome (SIRS), which encompass multiple etiologies (fibrin deposition, platelet aggregation, coagulopathies, and leukocyte lysosomal release). Manifestations of SIRS include abnormally high or low body temperature, elevated heart rate, high respiratory rate, and abnormal white blood cell counts. A systemic inflammation may be an infective inflammation or a sterile inflammation (sterile systemic inflammation).

By “infective” inflammation is meant an inflammation that is caused by an infection of a microbial pathogen such as a bacterium, virus, or fungus. An infective inflammation may be indicated by a microbial (e.g., bacterial) nucleic acid (e.g., 16S DNA or 16S rRNA) concentration of >0.5 μg/mL or >1 μg/mL.

By “sterile inflammation” is meant an inflammation that is not caused by an infection of a pathogen such as a bacterium, virus, or fungus. Causes of sterile infection include, for example, mitochondrial nucleic acid released from cells as a result of trauma. A subject may have an inflammation that has both “infective” and “sterile” etiologies. A sterile inflammation may be indicated by a mitochondrial nucleic acid (cytochrome B mitochondrial DNA) of >1 μg/mL or >0.5 μg/mL. A sterile inflammation may also be indicated by a mitochondrial nucleic acid to microbial (e.g., bacterial) nucleic acid ratio of >1:1000 or >1:800.

By “surgery” is meant an invasive therapeutic procedure. Non-limiting examples of surgery include elective surgery, emergency surgery, exploratory surgery, amputation, replantation, reconstructive surgery, cosmetic surgery, transplantation, angioplastic surgery, laparoscopic surgery, laparotomy, laser surgery, and microsurgery.

By “tissue damage” is meant cellular damage to a tissue in the body of a subject. Tissue damage may occur as a result of blunt trauma, may be induced by one or more (e.g., two, three, or four) disease states in a subject (e.g., hypotension, hypofusion/reperfusion injury, pancreatitis, and shock), or may be induced by therapeutic treatment (e.g., surgery or chemotherapy). Tissue damage may also be caused by a chronic disease state in a subject.

Other features and advantages of the invention will be apparent from the following description of the preferred embodiments thereof, and from the claims.

### BRIEF DESCRIPTION OF THE DRAWINGS

FIG. 1. Clinical trauma results in circulation of mitochondrial DAMPs. A-C: Real-time PCR results for mtDNA of plasma from 15 major trauma patients and 12 matched volunteers. mtDNA appeared at far fewer cycles in patients (Cyt B, 24.4±0.9 vs. 32.8±0.9; COX III, 22.8±0.5 vs. 31.1±0.9; NADH, 22.7±0.5 vs. 30.9±0.8). All data are reported as mean±s.e.m. D: mtDNA concentrations were determined for Cyt B in patients and volunteers (n=12). * p<0.05 (t-test). The data for each patient is shown as a different color.

FIG. 2. mtDNA circulating 24 hours after injury and in fracture fluids. A: mtDNA was measured by qPCR in plasma from patients with ISS>25 sampled 24 hours after trauma and matched volunteers (n≧10, *p<0.01, t-test). 1/Ct denotes the reciprocal of the count at which the sequence was detected: a direct function of concentration. B: mtDNA was measured by qPCR in the supernatant of reamings from femur fractures obtained at the time of clinical fracture repair (n≧10, *p<0.01, ANOVA/Dunn). qPCR for bacterial 16S rRNA showed there was no bacterial contamination of specimens.

FIG. 3. PMN [Ca²⁺]ᵢ responses to MTD. Rhabdomyosarcoma-derived MTD induces Ca²⁺ store depletion (A) and influx (A, B) in human PMN (n≧3, *p=0.01, t-test). C: PMN exposed to MTD then GRO-α exhibit heterologous desensitization. D: PMN stimulated with GRO-α or MTD subsequently challenged with ionomycin. E: PMN showed no response to MTD when challenged with a second dose. F: Responses to platelet activating factor (PAF) in PMN from MTD-injected rats. Traces with error bars were compiled from ≧3 experiments, others are exemplary. Traces may be displaced temporally for clarity.

FIG. 4. Potency of MTD from differing sources. A: MTD derived from liver, femur fractures, and skeletal muscle produced similar PMN calcium fluxes at the same dilution, approximating the response to 1 nM fMLF at ˜ 1/100 dilution. B: The calcium response to intact mitochondria is approximately the sum of the responses to the supernatants and pellets produced by sonication and centrifugation. All traces are the means of 3 experiments. Some traces are displaced for ease of viewing.

FIG. 5. MTD and calcium mobilization. A: CsH specifically inhibits FPR-induced calcium depletion in human PMN. B: In FIG. 2A, the data demonstrate that anti-FPR1 inhibition of calcium flux in response to MTD was essentially complete. The data in FIG. 5B show that the isotype control antibodies for the FIG. 2A experiment (anti-FPRL-1, anti-MMP-2) have no significant effects on responses to MTD. C: 1 μM ATP mobilizes PMN [Ca²⁺]ᵢ. Treatment of ATP with apyrase destroys ATP, resulting in a total absence of Ca²⁺ signaling. MTD causes the expected [Ca²⁺]ᵢ signal but its effect is resistant to apyrase. Some traces are displaced for visibility.

FIG. 6. MTD homologous desensitization. PMN with sequentially applied MTD-fMLF or fMLF-MTD showed completely inhibited Ca²⁺ response to the second stimuli. Traces are means of four experiments and are displaced for ease of viewing.

FIG. 7. MTD activate PMN. A-D: PMN were exposed to human muscle-derived MTD. Phosphorylated p38 (A) and p44/42 MAPK (B) were immunoblotted with total protein shown as controls. Panels C and D are from the same gel. αFPR1 denotes anti-FPR1. E-F: PMN IL-8 synthesis (mean±s.e.m., n=3/condition). *p<0.05 compared with control (ANOVA/Tukey's). **p<0.05 compared with control (ANOVA/Holm-Sidak). ***p<0.05 compared to either control or MTD (ANOVA/Holm-Sidak).

FIG. 8. MTD are potent PMN chemoattractants. Human PMN chemotaxis to MTD was analyzed by video-microscopy. PMN normally migrate briskly toward fMLF or MTD (A, B). Pre-treatment with CsH or anti-FPR1 (C, D) drastically inhibited chemotactic speed and directionality. E: Injection of MTD into the mouse peritoneal cavity causes rapid influx of neutrophils (n=6, *p<0.05, ANOVA/Dunn) compared with saline control, 10 nM W-peptide, or CsH inhibition.

FIG. 9. mtDNA activates PMN via CpG/TLR9 interaction. A: PMN (10⁶) were incubated with 1 μg/ml mtDNA for indicated times (n=3, *p<0.05 vs. unstimulated control, t-test). B: mtDNA-induced activation of p38 MAPK is inhibited by pre-incubation with the inhibitory ODN TTAGGG. Inhibition was overcome at higher mtDNA concentration. C: PMN were co-incubated in 1 nM fMLF and mtDNA at clinical concentrations (1-10 μg/ml; FIG. 1D). Neither CpG DNA nor mtDNA caused IL-8 release alone, but each caused significant release with low dose fMLF (n=3, *p<0.05 compared with unstimulated control, ANOVA/Dunn).

FIG. 10. PMN chemotaxis in transwell assay. PMN shows chemotaxis to fMLF (10 nM), MTD (100 μg/ml), and IL-8 (10 nM). CsH (1 μM) significantly inhibits PMN migrating to fMLF and MTD, but not IL-8. PMN shows no chemotaxis to mtDNA (10 μg/ml), CpG DNA (10 μg/ml), and LPS (1 μg/ml) (n=3,*p<0.05, t-test).

FIG. 11. MTD cause systemic inflammation and organ injury in vivo. Rats given MTD equivalent to a 5% liver injury i.v. show marked histologic evidence of ALI by hematoxylin and eosin staining (10×) (A-B) and 4-hydroxy-2-nonenal (4-HNE) staining (20×) (C-D). MTD increases pulmonary albumin permeability (E), lung wet/dry weight (F), PMN infiltration into the airways (G) and accumulation of IL-6 in lung (H). Early (3 hour) appearance of TNF-α (i) and late (6 hour) appearance of IL-6 (J) were noted in lung lavage fluid (G). Whole lung (K) and liver (L) MMP-8 confirmed increased PMN infiltration (n≧3, *p<0.05, ANOVA with post-hoc tests).

FIG. 12. PMN elastase in rat lung. Rats with MTD injection showed increased levels of elastase in lung tissue (n≧3 for each group of rats, *p<0.05, ANOVA/Holm-Sidak).

FIG. 13. Amplification of bacterial and mitochondrial DNA samples with cytochrome B primers shows specificity to mitochondrial DNA.

FIG. 14. Amplification of bacterial and mitochondrial DNA samples with 16s ribosomal RNA primers shows specificity to bacterial DNA and sensitivity to various bacteria types.

FIG. 15. Shows the sensitivity range of cytochrome B primers.

FIG. 16. Shows the sensitivity range of 16s ribosomal RNA primers.

FIG. 17. mtDNA and nDNA in trauma/hemorrhagic (T/HS) rat plasma. A: mtDNA was quantified in rat plasma by real-time PCR from rat liver using Cyt B as a target. Identical results were found using primers for other mitochondrial proteins: COX III and NADH (data not shown). T/HS significantly elevated plasma mtDNA level and the increased mtDNA levels last at least one week (*p<0.001 compared with naïve, ANOVA/Holm-Sidak). B: nDNA in T/HS rat plasma was assessed by real time PCR for GAPDH. The level of nDNA was rapidly increased, becoming significant at 3 hours after the end of T/HS. Levels gradually returned to normal (*p<0.05, ANOVA/Holm-Sidak) (Ct: threshold level).

FIG. 18. Effects of mtDNA on PMN MAPK activation. Human PMN were incubated with or without mtDNA at 1 and 5 μg/mL, with or without pre-incubation with chloroquine (10 μg/mL, 30 minutes, 37° C.). Phosphorylation of p38 and p44/42 MAPK was measured in PMN lysates. Total p38 and p44/42 are shown as protein loading controls. mtDNA activates p38, but not p44/42 MAPK. p38 MAPK responses to mtDNA at these doses were markedly inhibited by chloroquine.

FIG. 19. Effects of mtDNA on PMN degranulation. Representative bands are shown of Western blots of PMN equivalents (for degranulation of MMP-8/9) and of cell lysates (for p38 and p44/42 MAPK; n=3 for the indicated conditions). Human PMN were incubated with 100 μg/mL mtDNA (left panel) or 100 μg/mL nDNA (right panel) with or without pre-incubation in chloroquine (10 μg/mL). Total p38 and p44/42 are shown as controls indicating equal protein loading. mtDNA but not nDNA caused MMPs release and activation of p38 MAPK. As at the lower dose, p44/42 MAPK was not activated. Phosphorylation of p38 MAPK in response to mtDNA at these concentrations was not inhibited by chloroquine.

FIG. 20. Circulation of DNA after injection of MTD. A: mtDNA was quantified in rat plasma by real-time PCR using a standard curve of purified mtDNA. mtDNA levels were markedly elevated after MTD injection (*p=0.002, ANOVE/Dunn) compared with plasma from naïve or vehicle injected animals. B: nDNA was studies in the same samples, and there was almost no detectable nDNA in any of the groups.

FIG. 21. Mitochondrial debris (MTD) induces hepatic inflammation in vivo. A: Mitochondrial debris (MTD) activated p38 MAPK in lever. Rats were injected with a preparation of isolated, sonicated mitochondria and sacrificed at 1 hour after injection. p38 MAPK phosphorylation was evaluated by Western blot in whole liver homogenates. Representative bands are shown and the densitometry data is presented as mean±s.e (*p<0.05, ANOVA/Holm-Sidak). Injected animals showed marked increases in p38 MAPK phosphorylation compared with naïve or vehicle group. B: IL-6 was measured in whole liver homogenates by ELISA. IL-6 levels were significantly elevated in the MTD-injection group (**p<0.05, ANOVA/Dunn). C: TNF-α was measured in whole liver homogenates by ELISA. TNF-α levels were significantly elevated in the MTD-injection group compared with the naïve and vehicle groups (***p<0.05, ANOVA/Holm-Sidak).

FIG. 22. mtDNA is released into the pancreatic fluid during pancreatitis. The amount of mtDNA and bacterial DNA in pancreatic fluid in control rats and a rat model of pancreatitis were determined using real-time PCR. The fold-increase in mtDNA and bacterial DNA in pancreatic fluid relative to control is depicted.

### DETAILED DESCRIPTION

Trauma is the leading cause of premature death in the United States. Most preventable trauma death and morbidity is linked to the Systemic Inflammatory Response Syndrome (SIRS), the clinical manifestation of globally activated innate immunity. SIRS renders injured patients “ill,” causing organ failure and susceptibility to secondary infections. SIRS may also be induced by other disease states and other sources of tissue damage (e.g., hypotension, hypofusion/reperfusion injury, chemotherapy, pancreatitis, and shock). Neutrophil (PMN) dysfunction is a critical component of SIRS. Since SIRS is common after both injury and infection, inflammation after trauma was long thought to reflect ‘translocation’ of gut bacteria into the circulation. This was disproven by sampling portal blood in trauma patients, however, gut ischemia may still cause inflammation after trauma. Injuries like crushes or bums however, cause SIRS without shock. Thus, the molecular signals linking tissue injury to inflammation remain unclear.

Traumatic SIRS can be indistinguishable from sepsis, where innate immunity is activated via pathogen-associated molecular patterns (PAMPs) expressed on microorganisms. PAMPs are recognized by pattern recognition receptors (PRR). These are germline-defined sensors for a wide spectrum of molecular patterns that identify invading microorganisms. For instance, prokaryotic protein synthesis is initiated by N-formyl methionine. Thus N-formyl peptides activate formyl peptide receptors (FPR) and are potent chemoattractants for human neutrophils (PMN). Immune cells also express Toll-like receptors (TLR) that bind and respond to bacterial motifs. For example, bacterial DNA is circular and high in CpG repeats, allowing recognition by TLR9.

Although eukaryotic somatic proteins are not N-formylated, mitochondria closely resemble bacteria in that they contain circular CpG DNA (mtDNA) and formylated peptides. These observations show that mitochondria were once saprophytic organisms that evolved into endosymbionts and finally, intracellular organelles with a genome that codes for only thirteen proteins, each N-formylated. Mitochondria are intracellular, but in blunt trauma, billions of cells can be instantaneously disrupted or rapidly rendered necrotic. Other types of tissue damage (e.g., induced by a disease state or therapeutic treatment) may also result in a release of mtDNA into the blood. Despite the similarities of mitochondria to bacteria and of sepsis to SIRS, the possibility that mitochondria might contain damage-associated molecular patterns (DAMPs) that activate innate immunity and cause SIRS has been unstudied. We have discovered that tissue damage (e.g., blunt trauma and tissue damage resulting from a disease state or therapeutic treatment) mobilizes mitochondrial DAMPs (MTD) into the circulation, initiating neutrophil activation via PRR and thus initiating SIRS and organ injury. In view of this discovery, methods for predicting the likelihood that a subject will develop a sterile inflammation, identifying a subject with an increased propensity to develop a sterile inflammation, and determining whether a subject with tissue damage should be administered an antimicrobial (e.g., antibacterial) agent or a reduced dosage of an antimicrobial (e.g., antibacterial) agent are provided. Also provided are methods of treating subjects with tissue damage.

### Diagnostic Methods

The likelihood of a subject (e.g., a human, cat, dog, horse, rabbit, mouse, monkey, or rat) to develop a sterile inflammation may be determined by: (a) measuring the amount of microbial (e.g., bacterial) nucleic acid or peptide in a sample from the subject; (b) measuring the amount of mitochondrial nucleic acid or peptide in the sample; and (c) determining whether the subject has an increased likelihood (e.g., at least a 5%, 10%, 15%, 20%, 25%, 30%, 35%, 40%, 45%, 50%, 55%, 60%, 65%, 70%, 75%, 80%, 85%, 90%, 95%, 100%, 110%, 120% 130%, 140%, 150%, 160%, 170%, 180%, 190%, 200%, 210%, 220%, 230%, 240% 250%, 260%, 270%, 280%, 290%, 300%, 350%, 400%, 450%, 500%, 550%, 600%, 650%, 700%, 750%, 800%, 850%, 900%, 950%, 1000%, 1100%, 1200%, 1300%, 1400%, 1500%, 1600%, 1700%, 1800%, 1900%, 2000%, 2100%, 2200%, 2300%, 2400%, 2500%, 2600%, 2700%, 2800%, 2900%, or 3000% increased likelihood) of later developing (e.g., at least 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 24 hours, 28 hours, 32 hours, 36 hours, 40 hours, 44 hours, 48 hours, 3 days, 4 days, 5 days, 6 days, or 7 days after tissue injury) a sterile inflammation (e.g., a sterile systemic inflammation) by comparing the amount of microbial (e.g., bacterial) nucleic acid or peptide measured in the sample with the amount of mitochondrial nucleic acid or peptide measured in the sample, where an increased ratio of the amount of mitochondrial nucleic acid or peptide to the amount of microbial (e.g., bacterial) nucleic acid or peptide indicates a subject with an increased likelihood of later developing a sterile inflammation.

Subjects may also be identified as having an increased propensity (e.g., at least a 5%, 10%, 15%, 20%, 25%, 30%, 35%, 40%, 45%, 50%, 55%, 60%, 65%, 70%, 75%, 80%, 85%, 90%, 95%, 100%, 110%, 120% 130%, 140%, 150%, 160%, 170%, 180%, 190%, 200%, 210%, 220%, 230%, 240% 250%, 260%, 270%, 280%, 290%, 300%, 350%, 400%, 450%, 500%, 550%, 600%, 650%, 700%, 750%, 800%, 850%, 900%, 950%, 1000%, 1100%, 1200%, 1300%, 1400%, 1500%, 1600%, 1700%, 1800%, 1900%, 2000%, 2100%, 2200%, 2300%, 2400%, 2500%, 2600%, 2700%, 2800%, 2900%, or 3000% increased propensity) to later develop at least 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 24 hours, 28 hours, 32 hours, 36 hours, 40 hours, 44 hours, 48 hours, 3 days, 4 days, 5 days, 6 days, or 7 days after tissue injury) a sterile inflammation (e.g., a sterile systemic inflammation) by: measuring the amount of microbial (e.g., bacterial) nucleic acid or peptide in a sample from the subject; measuring the amount of a mitochondrial nucleic acid or peptide in the sample; and comparing the amount of microbial (e.g., bacterial) nucleic acid or peptide measured with the amount of mitochondrial nucleic acid or peptide measured, where an increased ratio of the amount of mitochondrial nucleic acid or peptide to the amount of microbial (e.g., bacterial) nucleic acid or peptide identifies a subject as having an increased propensity to later develop a sterile inflammation.

In these methods, the subject may have previously experienced tissue damage (e.g., within 60 minutes, 90 minutes, 2 hours, 150 minutes, 3 hours, 3.5 hours, 4 hours, 4.5 hours, 5 hours, 5.5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 42 hours, 48 hours, 54 hours, 60 hours, 66 hours, or 72 hours). For example, the subject may have previously experienced blunt trauma (e.g., crushing, lacerations, and concussions). The subject may also have tissue damage resulting from a disease state (e.g., such as a chronic disease state) that has a duration of at least several days, several weeks, several months, or several years. For example, a chronic illness may result in a slow accumulation of tissue damage over at least three days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, or 5 years. Non-limiting examples of disease states that can induce tissue damage include hypotension, hypofusion/reperfusion injury, pancreatitis, and shock. The subject may also experience tissue damage as a result of receiving therapeutic treatment (e.g., surgery, femur reaming, and chemotherapy).

The sample may represent any specimen obtained from a subject. Non-limiting examples of a sample include blood, serum, plasma, urine, saliva, amniotic fluid, cerebrospinal fluid, tissue (e.g., placental or dermal), pancreatic fluid, chorionic villus sample, and cells taken from a subject. The sample may be taken from a portion of the body affected by sterile inflammation (e.g., a sterile systemic inflammation) (e.g., pancreatic fluid from a subject having pancreatitis). The sample may be obtained from a subject within 5 hours of tissue damage (e.g., within 4 hours, 3 hours, 2 hours, 1 hour, 30 minutes, and 15 minutes of tissue damage). The sample may be obtained by intravenous puncture, intraarterial puncture, lumbar puncture, amniopuncture, urine sample collection, sputum collection, or biopsy.

In the provided methods, the subject may also not have (present with) symptoms of a systemic inflammation Non-limiting examples of such symptoms include: altered body temperature (e.g., less than 36° C. or greater than 38° C.), increased heart rate (e.g., greater than 90 beats per minute), tachypnea (e.g., greater than 20 breaths per minute), decreased arterial pressure of CO₂ (e.g., less than 4.3 kPa), altered white blood count (e.g., less than 4,000 cells/mm³ or greater than 12,000 cells/mm³), increased histamine levels (e.g., greater than 60 ng/mL in blood), increased leukotriene B4 levels (e.g., greater than 30 pg/mL or greater than 35 pg/mL in blood), increased prostaglandin levels (e.g., greater than 3.0 ng/mL in blood), increased levels of pro-inflammatory cytokines (e.g., greater than 20 ng/mL TNF-α and/or greater than 10 pg/mL IL-6).

The amount of mitochondrial nucleic acid or peptide present in a sample may be measured using standard techniques known in the art. For example, mitochondrial nucleic acids may be measured using quantitative techniques such as real-time qPCR using primers designed to specifically amplify nucleic acid sequences present in the mitochondrial genome. In desirable embodiments, the mitochondrial nucleic acid sequence amplified by qPCR is unique to the mitochondrial genome and is not present in the nuclear genome of the subject (e.g., a mammal). For example, mitochondrial nucleic acid sequences that may be measured in the above methods include cytochrome B, cytochrome C oxidase subunit III, and NADH dehydrogenase. For humans, amplification of mitochondrial cytochrome B may be performed using the forward primer 5′-atgaccccaatacgcaaaat-3′ (SEQ ID NO: 1) and the reverse primer 5′-cgaagtttcatcatgcggag-3′ (SEQ ID NO: 2), amplification of mitochondrial cytochrome C oxidase subunit III may be performed using the forward primer 5′-atgaccccaatacgcaaaat-3′ (SEQ ID NO: 3) and reverse primer 5′-cgaagtttcatcatgcggag-3′ (SEQ ID NO: 4) or the forward primer 5′-atgacccaccaatcacatgc-3′ (SEQ ID NO: 15) and the reverse primer 5′-atcacatggctaggccggag-3′ (SEQ ID NO: 16), and amplification of mitochondrial NADH dehydrogenase may be performed using the forward primer 5′-atacccatggccaacctcct-3′ (SEQ ID NO: 5) and the reverse primer 5′-gggcctttgcgtagttgtat-3′ (SEQ ID NO: 6). Additional primers may be used to amplify additional mitochondrial nucleic acid sequences, including but not limited to nucleic acid sequences containing a sequence at least 95% (e.g., at least 96%, 97%, 98%, 99%, or even 100% identical) to NADH dehydrogenase subunit I (nucleotides 3308-4264 of SEQ ID NO: 21), NADH dehydrogenase subunit II (nucleotides 4471-5512 of SEQ ID NO: 21), NADH dehydrogenase subunit III (nucleotides 10,060 to 10,405 of SEQ ID NO: 21), NADH dehydrogenase subunit IV (nucleotides 10,761 to 12,138 of SEQ ID NO: 21), NADH-ubiquinone oxidoreductase chain 4L (nucleotides 10,471 to 10,767 of SEQ ID NO: 21), NADH dehydrogenase subunit V (nucleotides 12,338 to 14,149 of SEQ ID NO: 21), NADH dehydrogenase VI (nucleotides 14,150 to 14,674 of SEQ ID NO: 21), cytochrome B (nucleotides 14,748 to 15,882 of SEQ ID NO: 21), cytochrome C oxidase subunit I (nucleotides 5905 to 7446 of SEQ ID NO: 21), cytochrome C oxidase subunit II (nucleotides 7587 to 8270 of SEQ ID NO: 21), cytochrome C oxidase subunit III (nucleotides 9208 to 9988 of SEQ ID NO: 21), ATP synthase F0 subunit VI (nucleotides 8528 to 9208 of SEQ ID NO: 21), and ATP synthase subunit VIII (nucleotides 8,367 to 8,573 of SEQ ID NO: 21). The levels of the measured mitochondrial nucleic acid samples may be normalized to a standard or a reference in the real-time PCR experiment. For example, a real-time PCR standard curve may be created to quantify the mtDNA concentration by using purified mtDNA. Methods for the isolation of mtDNA are described in the Examples. The threshold level (Ct) for amplification in real-time PCR may be set at 20, 25, 30, 35, or 40 cycles for statistical purposes. Desirably, the threshold level for amplification in real-time PCR is set at 30 or 40 cycles. Mitochondrial RNA may also be measured as the mitochondrial nucleic acid in the above methods. In such experiments, a first step of synthesis of a cDNA copy of the mitochondrial RNA is performed using reverse transcriptase prior to amplification in a real-time PCR experiment.

Mitochondrial peptides (N-formylated peptides) may be measured using standard methods known in the art. For example, expression of mitochondrial proteins (e.g., NADH dehydrogenase subunit I, NADH dehydrogenase subunit II, NADH dehydrogenase subunit III, NADH dehydrogenase subunit IV, NADH-ubiquinone oxidoreductase chain 4L, NADH dehydrogenase subunit V, NADH dehydrogenase subunit VI, cytochrome B, cytochrome C oxidase subunit I, cytochrome C oxidase subunit II, cytochrome C oxidase subunit III, ATP synthase F0 subunit VI, and ATP synthase subunit VIII) may be measured using ELISA assays, Western blotting assays, or protein array assays. The relative level of expression of mitochondrial proteins may be compared to the levels of purified mitochondrial proteins or to other control proteins present in the sample. A number of antibodies that specifically bind mitochondrial peptides are commercially available.

Similarly, the amount of microbial (e.g., bacterial, fungal, or viral) nucleic acids or peptides in a sample may be measured using standard techniques known in the art. For example, bacterial nucleic acids may be measured quantitative techniques such as real-time PCR using primers designed to specifically amplify sequences present in the bacterial genome. In desirable embodiments, the bacterial nucleic acid that is amplified is common to all species of bacteria, but not expressed in a mammalian cell. For example, specific sequences in 16S rRNA are conserved among many bacterial species and may be used to design primers that amplify 16S rRNA from several different species of bacteria using real-time PCR. In other examples, the primers used to quantitate the bacterial nucleic acid are designed to amplify 16S rRNA from a single species of bacteria using real-time PCR (see, for e.g., the primers described in WO 08/03957, herein incorporated by reference). In desirable embodiments, the bacterial nucleic acid sequence amplified by real-time PCR is unique to bacteria and is not expressed in a mammalian cell. One set of primers that may be used to amplify 16S rRNA from a variety of bacterial species are 5′-cgtcagctcgtgttgtgaaa-3′ (SEQ ID NO: 13) and 5′-ggcagtctccttgagttcc-3′ (SEQ ID NO: 14). The levels of the measured bacterial nucleic acid may be normalized to a standard or reference in the real-time PCR experiment. For example, a real-time PCR standard curve may be created to quantify the bacterial nucleic acid concentration by using purified 16S rRNA. Methods for the isolation of 16S rRNA for use as a standard control are known in the art. The threshold level (Ct) for amplification in real-time PCR may be set at 20, 25, 30, 35, or 40 cycles for statistical purposes. Desirably, the threshold level (Ct) for amplification in real-time PCR is set at least 20 or at least 30 cycles. As noted above, prior to direct use in real-time PCR, 16S rRNA or bacterial RNA must first be reverse transcribed into a cDNA prior to its amplification in real-time PCR. Similarly, primers for use in real-time PCR may be designed to amplify sequences present in several species of fungi, specific species of fungi, a family of viruses (e.g., influenza viruses), or specific virus strains (e.g., H1N1 influenza virus). Sequences for several fungi and viruses are known in the art.

The sample obtained from the subject may need to be treated in order to release the microbial (e.g., bacterial, fungal, or viral) DNA from any microorganisms (e.g., bacteria, fungi, or viruses) present in the sample. For example, methods for the use of a microfluidic device for lysis of bacterial cells in a sample are described in WO 09/002580 and U.S. 2007/0015179, incorporated by reference in its entirety. Additional methods for bacterial lysis in a biological sample are known in the art and include without limitation: alkaline lysis (provided in a number of commercially available kits), lysozyme treatment, physical disruption (e.g., French press), or combination thereof. Such lysis methods may be used prior to the subsequent amplification of the nucleic acids using PCR-based techniques (e.g., real-time PCR).

Microbial (e.g., bacterial) peptides may also be measured using standard methods known in the art. For example, expression of microbial (e.g., bacterial, fungal, or viral) proteins may be measured using ELISA assays, Western blotting assays, or protein array assays. The relative level of expression of microbial (e.g., bacterial) proteins may be compared to the levels of purified microbial (e.g., bacterial) proteins or to other control proteins present in the sample. A number of antibodies that specifically bind microbial (e.g., bacterial, fungal, or viral) peptides are commercially available.

A ratio (increased ratio) of the amount of mitochondrial nucleic acid or peptide to the amount of microbial (e.g., bacterial) nucleic acid or peptide present in a sample that indicates that a patient having an increased likelihood or propensity of later developing a sterile inflammation may be a ratio of at least 5.0:1, 6.0:1, 7.0:1, 8.0:1, 9.0:1, 10:1, 11:1, 12:1, 13:1, 14:1, 15:1, 16:1, 17:1, 18:1, 19:1, 20:1, 25:1, 26:1, 27:1, 28:1, 29:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, 1600:1, 1700:1, 1800:1, 1900:1, or 2000:1. The term increased ratio may be compared related to a threshold ratio (e.g., one of the ratios listed above) or the measured ratio in a control subject (e.g., a subject without tissue damage or not suffering from a disease state). The determined ratio may represent a mass ratio or a molar ratio of the nucleic acids or proteins. For a raw ratio obtained from a measured nucleic acid, the amounts may be normalized in various ways (e.g., for relative nucleic acid lengths, amplification biases, and other experimental considerations), before it is assessed against a cutoff value or used to determine a confidence level or to calculate the ratio. Similarly, for a raw ratio obtained from a measured protein, the amounts may be normalized to a another protein present in the sample (e.g., normalized to the level of a house-keeping gene such as actin), before it is assessed against a cutoff value or used to determine a confidence level or to calculate the ratio.

Also provided are methods of determining whether a systemic inflammation in a subject is an infective inflammation, a sterile inflammation, or both, that require the steps of: (a) measuring the amount of a microbial (e.g., bacterial) nucleic acid or peptide in a sample derived from the subject; (b) measuring the amount of a mitochondrial nucleic acid or peptide in the sample; and (c) determining whether the systemic inflammation is an infective inflammation, a sterile inflammation, or both based on the measured amounts of microbial (e.g., bacterial) nucleic acid or peptide and mitochondrial nucleic acid or peptide. The amount of a mitochondrial nucleic acid or peptide and the amount of a microbial (e.g., bacterial) nucleic acid or peptide may be measured as described above. A systemic inflammation that is an infective inflammation may be indicated by a ratio of mitochondrial nucleic acid or peptide to microbial (e.g., bacterial) nucleic acid or peptide of less than 5:1, 4:1, 3:1, 2:1, 1:1, 0.5:1, 0.1:1. 0.01:1, or 0.005:1, or 0.001:1. A systemic inflammation that is a sterile inflammation may be indicated by a ratio of mitochondrial nucleic acid or peptide to microbial (e.g., bacterial) nucleic acid or peptide of 5.0:1, 6.0:1, 7.0:1, 8.0:1, 9.0:1, 10:1, 11:1, 12:1, 13:1, 14:1, 15:1, 16:1, 17:1, 18:1, 19:1, 20:1, 25:1, 26:1, 27:1, 28:1, 29:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, 1600:1, 1700:1, 1800:1, 1900:1, or 2000:1. A systemic inflammation that is both a sterile inflammation and an infective inflammation may be indicated by a ratio of mitochondrial nucleic acid or peptide to microbial (e.g., bacterial) nucleic acid or peptide of: 50:1, 40:1, 30:1, 20:1, 15:1, 10:1, 5:1, 3:1, 2:1, 1:1, 0.5:1, 0.1:1, 0.2:1, 0.1:1, 0.01:1, or 0.005:1.

The ratio of mitochondrial nucleic acid or peptide and bacterial nucleic acid and peptide may vary progressively with the relative severity of sterile and/or infective inflammation. An infective inflammation may be indicated, for example, by a microbial (e.g., bacterial) nucleic acid concentration of >1 μg/mL or >0.5 μg/mL in a sample from a subject. In infections (e.g., bacterial infections) where virulence is due to invasiveness, an infective inflammation may be indicated, for example, by a microbial (e.g., bacterial) nucleic acid concentration of greater than >2 μg/mL, >3 μg/mL, or >5 μg/mL. In infections where virulence contributed in part to a toxin (e.g., a bacterial endotoxin), an infective inflammation may be indicated, for example, by a microbial (e.g., bacterial) nucleic acid ratio of greater than >0.8 μg/mL, >0.5 μg/mL, or >0.1 μg/mL.

A sterile inflammation may also be indicated, for example, by a mitochondrial nucleic acid (e.g., cytochrome B DNA) concentration in the sample of >1 μg/mL or >0.5 μg/mL.

In non-limiting examples of the provided methods, a ratio of mitochondrial nucleic acid or peptide to microbial (e.g., bacterial) nucleic acid or peptide of >1000:1, >800:1, or >500:1 may indicate a sterile inflammation. As noted above, this ratio may vary based on the infectious microbial species (e.g., bacterial species releasing a toxin may have a lower ratio, while bacterial species that are highly invasive may have a higher ratio).

The statistical confidence with which a determination of sterile versus infective inflammation may be made will vary based on the measured mitochondrial nucleic acid or peptide to microbial (e.g., bacterial) nucleic acid or peptide ratio. For example, sterile inflammation may be indicated by a ratio of >1000:1, >800:1, or >500:1 and/or the absence of microbial (e.g., bacterial) nucleic acid or peptide. The absence of an infective inflammation may also be indicated by a microbial (e.g., bacterial) nucleic acid concentration of <1 pg/mL (reliable indication of absence of infective inflammation) or <1 ng/mL (highly reliable indication of absence of infective inflammation) in the sample.

### Methods of Treatment

The invention further provides methods of determining whether a subject with tissue damage should be administered an antimicrobial (e.g., antibacterial) agent or a reduced dosage (e.g., a reduction in the standard dose of a antimicrobial (e.g., antibacterial) agent by at least 5%, 10%, 15%, 20%, 25%, 30%, 35%, 40%, 45%, 50%, 55%, 60%, 65%, 70%, 75%, 80%, 85%, 90%, or 95%) by: measuring the amount of microbial (e.g., bacterial) nucleic acid or peptide in a sample from the subject; measuring the amount of mitochondrial nucleic acid or peptide in the sample; and comparing the amount of microbial (e.g., bacterial) nucleic acid or peptide measured to the amount of mitochondrial nucleic acid or peptide measured, where a subject having an increased ratio of the amount of mitochondrial nucleic acid or peptide to the amount of microbial (e.g., bacterial) nucleic acid or peptide should not be administered an antimicrobial (e.g., antibacterial) agent or administered a reduced amount of an antimicrobial (e.g., antibacterial) agent.

In addition, methods of treating a subject with tissue damage are provided that require: measuring the amount of microbial (e.g., bacterial) nucleic acid or peptide in a sample from the subject; measuring the amount of a mitochondrial nucleic acid or peptide in the sample; comparing the amount of microbial (e.g., bacterial) nucleic acid or peptide measured to the amount of mitochondrial nucleic acid or peptide; and administering to the subject having an increased ratio of the amount of mitochondrial nucleic acid or peptide one or more anti-inflammatory agents (e.g., cyclosporin H, anti-FPR antibodies, CpG oligodeoxynucleotides (e.g., CpG oligonucleotides containing at least one modified nucleotide monomer, such as LNA), chloroquin, and/or anti-TLR9 antibodies) and not administered, or administered at a reduced dosage (e.g., a reduction in the standard dose of at least 5%, 10%, 15%, 20%, 25%, 30%, 35%, 40%, 45%, 50%, 55%, 60%, 65%, 70%, 75%, 80%, 85%, 90%, or 95%), an antimicrobial (e.g., antibacterial) agent.

The patients treated with these methods may be any of the subject populations described above (e.g., subjects with tissue damage resulting from blunt trauma or tissue damage resulting from a disease state or therapeutic treatment).

A variety of different samples may be obtained from the subjects using any of the above described methods. The sample may be obtained from the subject at a variety of different time points as described above (e.g., within different periods of time following tissue damage or after accumulation of tissue damage resulting from a disease state over at least three days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, or 5 years).

A ratio (increased ratio) of the amount of mitochondrial nucleic acid or peptide to the amount of microbial (e.g., bacterial) nucleic acid or peptide present in a sample that indicates that a patient should not be administered an antimicrobial (e.g., antibacterial) agent or a reduced dosage of an antimicrobial (e.g., antibacterial) agent and/or should be administered one or more (e.g., two, three, four, or five) anti-inflammatory agents (e.g., cyclosporin H, anti-FPR antibodies, CpG oligodeoxynucleotides, chloroquin, and/or anti-TLR9 antibodies) may be a ratio of at least 5.0:1, 6.0:1, 7.0:1, 8.0:1, 9.0:1, 10:1, 11:1, 12:1, 13:1, 14:1, 15:1, 16:1, 17:1, 18:1, 19:1, 20:1, 25:1, 26:1, 27:1, 28:1, 29:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 250:1, 300:1, 350:1, 400:1, 450:1, 500:1, 550:1, 600:1, 650:1, 700:L 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, 1600:1, 1700:1, 1800:1, 1900:1, or 2000:1. The term increased ratio may be compared related to a threshold ratio (e.g., one of the ratios listed above) or the measured ratio in a control subject (e.g., a subject without tissue damage or not suffering from a disease state). The determined ratio may represent a mass ratio or a molar ratio of the nucleic acids or proteins. For a raw ratio obtained from a measured nucleic acid, the amounts may be normalized in various ways (e.g., for relative nucleic acid lengths, amplification biases, and other experimental considerations), before it is assessed against a cutoff value or used to determine a confidence level or to calculate the ratio. Similarly, for a raw ratio obtained from a measured protein, the amounts may be normalized to another protein present in the sample (e.g., normalized to the level of a house-keeping gene such as β-actin), before it is assessed against a cutoff value or used to determine a confidence level or to calculate the ratio. All of the methods for measuring the amount of a microbial (e.g., bacterial) nucleic acid or peptide and the amount of a mitochondrial nucleic acid or peptide described above may be used in the treatment methods without limitation.

The invention further provides methods of administering to a subject one or more (e.g., two, three, four, or five) anti-inflammatory agents (cyclosporin H, anti-FPR antibodies, CpG oligodeoxynucleotides, chloroquin, and/or anti-TLR9 antibodies) to a subject indicated as having an increased propensity to later develop a sterile inflammation.

Non-limiting examples of antimicrobial agents (e.g., antibacterial, anti-fungal, and/or anti-protozoan agents) that may not be administered or administered at a decreased dosage include: amikacin, gentamycin, kanamycin, neomycin, netilmicin, tobramycin, paromomycin, geldanamycin, herbimycin, loracarbef, ertapenem, doripenem, imipenem, meropenem, cefadroxil, cefazolin, cefalotin, cefalexin, cefaclor, cefamandole, cefoxitin, cefprozil, cefuroxime, cefixime, cefdinir, cefditoren, cefotaxime, cefpodoxime, ceftazidime, ceftibuten, ceftizoxime, ceftriaxone, cefepime, ceftobiprole, teicoplanin, vancomycin, telavancin, clindamycin, lincomycin, azithromycin, clarithromycin, dirithromycin, erythromycin, roxithromycin, troleandomycin, telithromycin, telithromycin, spectinomycin, aztronam, furazolidone, nitrofurantoin, nitrofurantoin, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, bacitracin, colistin, polymyxin B, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfonamidochrysoidine, sulfacetamide, sulfadiazine, silver sulfadiazine, sulfamethizole, sulfamethoxazole, sulfanilamide, sulfasalazine, sulfisoxazole, trimethoprim, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, choramphenicol, fosfomycin, fusidic acid, linezolid, metronidazole, mupirocin, platensimycin, quinupristin, rifaximin, thiamphenicol, and tinidazole.

Anti-inflammatory agents that may be administered in the methods of treatment include without limitation: ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lomoxicam, isoxicam, mefenaic acid, meclofenamic acid, tolfenamic acid, celecoxib, rofecoxib, valdecoxib, parecoxib, lumiracoxib, etoricoxib, firoxocib, nimesulide, immunosuppressive agents (e.g., methotrexate, azathioprine, basiliximab, daclizumab, cyclosporine, tacrolimus, sirolimus, voclosporin, infliximab, etanercept, adalimumab, mycophenolic acid, fingolimod, pimecrolimus, thalidomide, lenalidomide, anakinra, deferolimus, everolimus, temsirolimus, zotarolimus, biolimus A9, and elsilimomab), corticosteroids (e.g., hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate), cyclosporin H, anti-FPR antibodies, CpG oligodeoxynucleotides (e.g., CpG oligodeoxynucleotides containing one or more modified nucleotides, such as LNA), chloroquin, and anti-TLR9 antibodies.

One or more anti-inflammatory agent(s) may be administered to the subject at a dose of 0.1 mg to 10 mg, 1 mg to 50 mg, 1 mg to 100 mg, 50 mg to 100 mg, 50 mg to 200 mg, 100 mg to 200 mg, 100 mg to 500 mg, 250 mg to 500 mg, 400 mg to 800 mg, 500 mg to 1 g, 600 mg to 1.5 g, 800 mg to 1.2 g, 1.0 g to 1.5 g, 1.5 g to 2.0 g. The amount and frequency of administration will dependent on several factors that may be determined by a physician including the mass, sex, disease state, and age of the subject. For example, a subject may be administered one or more anti-inflammatory agents continuously, every 2 hours, every 3 hours, every 4 hours, every 5 hours, every 6 hours, every 8 hours, every 10 hours, every 12 hours, once a day, two times a day, three times a day, four times a day, once a week, twice a week, three times a week, four times a week, five times a week, six times a week, seven times a week, biweekly, monthly, or bimonthly. The one or more anti-inflammatory agent(s) may be administered by any known means of administration, e.g., orally, intravenously, subcutaneously, and intaarterially. The subject may be monitored by a physician during the treatment for the development of symptoms of a systemic inflammation. In response to the development of such symptoms, the physician may administer an increased dosage of one or more anti-inflammatory agents or increase the frequency of administration of such anti-inflammatory agents.

### Kits

The invention also provides kits containing one or more (e.g., two, four, six, or eight) oligonucleotide primers effective (e.g., capable of hybridizing to a bacterial nucleic acid) for the amplification of a microbial (e.g., bacterial, fungal, or viral) nucleic acid, one or more (e.g., two, four, six, or eight) oligonucleotide primers effective (e.g., capable of hybridizing to a mitochondrial nucleic acid) for the amplification of mitochondrial nucleic acids, and instructions for using these primers to determine the likelihood that a subject will develop a sterile inflammation, to identify a subject that has an increased propensity to later develop a sterile inflammation, and to determine whether a subject with tissue damage should be administered an antimicrobial (e.g., antibacterial) agent or a reduced dosage of an antimicrobial (e.g., antimicrobial) agent. These kits may include, without limitation, any of the nucleic acid primers described above for use in the diagnostic methods. The kits may further include control nucleic acid sequences for use in real-time qPCR including, but not limited to, purified mtDNA and/or bacterial 16S rRNA. The instructions provided with the kits may describe how to calculate the specific ratio of the amount of mitochondrial nucleic acid to the amount of microbial (e.g., bacterial) nucleic acid (e.g., exemplary methods for the calculation of the ratio are described herein). The instructions may also describe the comparison of the calculated ratio to a specific threshold value or a ratio measured from a control sample (e.g., a subject not having tissue damage or a disease state).

The invention also provides kits containing one or more (e.g., two, three, or four) antibodies that specifically bind one or more microbial (e.g., bacterial, fungal, or viral) peptides, one or more (e.g., two, three, or four) antibodies that specifically bind one or more mitochondrial peptides, and instructions for using these antibodies to determine the likelihood that a subject will develop a sterile inflammation, to identify a subject that has an increased propensity to later develop a sterile inflammation, and to determine whether a subject with tissue damage should be administered an antimicrobial (e.g., antibacterial) agent or a reduced dosage of an antimicrobial (e.g., antimicrobial) agent. These kits may include, without limitation, any of antibodies described above for use in the diagnostic methods. The kits may further include control peptides for use in ELISA assays including, but not limited to, purified mtDNA and/or microbial peptides. The instructions provided with the kits may describe how to calculate the specific ratio of the amount of mitochondrial peptide to the amount of microbial peptide (e.g., exemplary methods for the calculation of the ratio are described herein). The instructions may also describe the comparison of the calculated ratio to a specific threshold value or a ratio measured from a control sample (e.g., a subject not having tissue damage or a disease state).

### EXAMPLES

The features and other details of the invention will now be more particularly described and pointed out in the following examples describing preferred techniques and experimental results. These examples are provided for the purpose of illustrating the invention and should not be construed as limiting.

### Example 1

### Clinical Trauma Releases Mitochondrial DAMPs into the Circulation

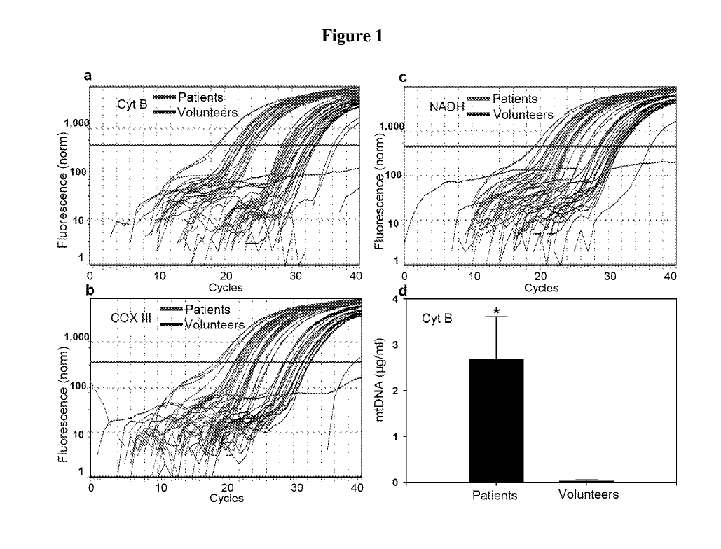

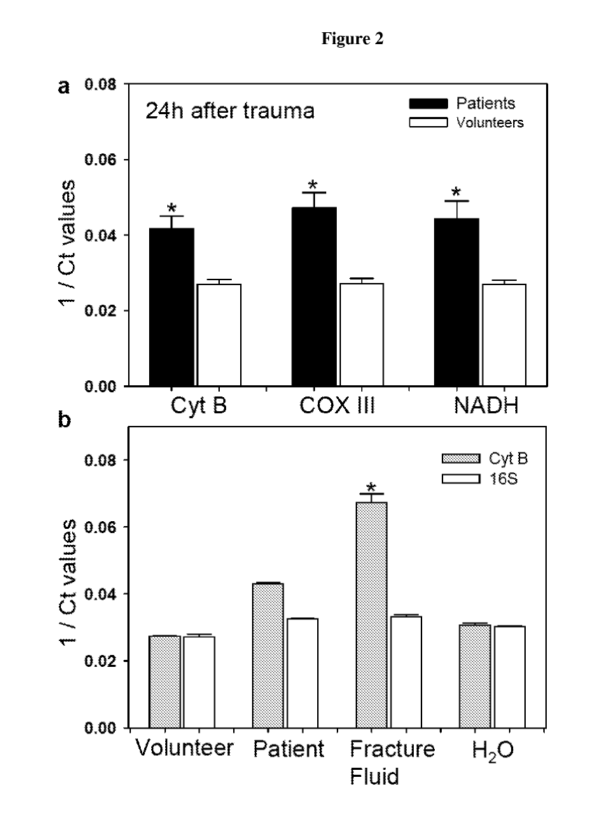

To demonstrate that major trauma releases MTD into the circulation, we used qPCR to measure plasma mtDNA obtained from 15 major trauma patients with Injury Severity Scores (ISS)>25. Samples were taken prior to resuscitation. Patients had no apparent open wounds or gastrointestinal injuries, excluding wound contamination and ischemia-reperfusion injury as sources of bacterial DNA (clinical details in Table 1). Major trauma markedly elevated plasma titers of mtDNA encoding cytochrome B (Cyt B, FIG. 1A), cytochrome C oxidase subunit III (COX III, FIG. 1B), and NADH dehydrogenase (NADH, FIG. 1C) when compared to healthy volunteer plasma (Table 2, n=12). These primer sequences have no significant homology with DNA found in any bacterial species published on BLAST. mtDNA concentration in trauma patient plasma was 2.7±0.94 μg/ml, where volunteer levels were a thousand times lower (FIG. 1D). Plasma mtDNA was still higher in trauma patient plasma 24 hours after injury (FIG. 2A). We also examined ultracentrifugation supernatants from femur reaming specimens obtained during clinical fracture repairs. These fluids contain cellular debris and had even higher titers of mtDNA. Thus MTD can be released into local tissues and the circulation either by accidental blunt trauma or by operative injury, with plasma mtDNA titers rising up to a level a thousand-fold greater than those found in healthy subjects. In addition, PCR for bacterial 16S ribosomal RNA demonstrated a lack of bacterial contamination in all specimen groups (FIG. 2B). These data show that wounds act as a source of DAMPs, entering the systemic circulation passively from mechanically disrupted cells and elevating mtDNA levels.

Table 1 shows the demographics and clinical details of the trauma patient population. Clinical data are reported for the injured patients with serum mtDNA measurements shown in FIG. 1. No patient had any significant medical co-morbidity (study exclusion). No patient had a major open injury or intestinal injury (study exclusion). Mean age was 43±21 years (SD, median 41 years). The mean time to blood sampling was 98±23 minutes (SD, median 93 minutes). AIS signifies the Abbreviated Injury Score for each specific body region, and is followed by the numeric score assigned that injury or group of injuries (0-5 points in each region). The ISS is the Injury Severity Score, which is defined as the sum of the squared scores of the three worst injured areas (0-75 points). Buffer base excess is calculated from arterial blood gas pH and pCO₂, and is an indicator of tissue perfusion.

Table 2: Demographics of volunteer control blood donors. Clinical data is presented for the volunteers with serum mtDNA measurements shown in FIG. 1. No patient had an acute injury. Two subjects had chronic uncomplicated type II diabetes. Mean age was 41±14 (SD, median 36) years.

### Mitochondrial DAMPs Induce Inflammatory PMN Signaling

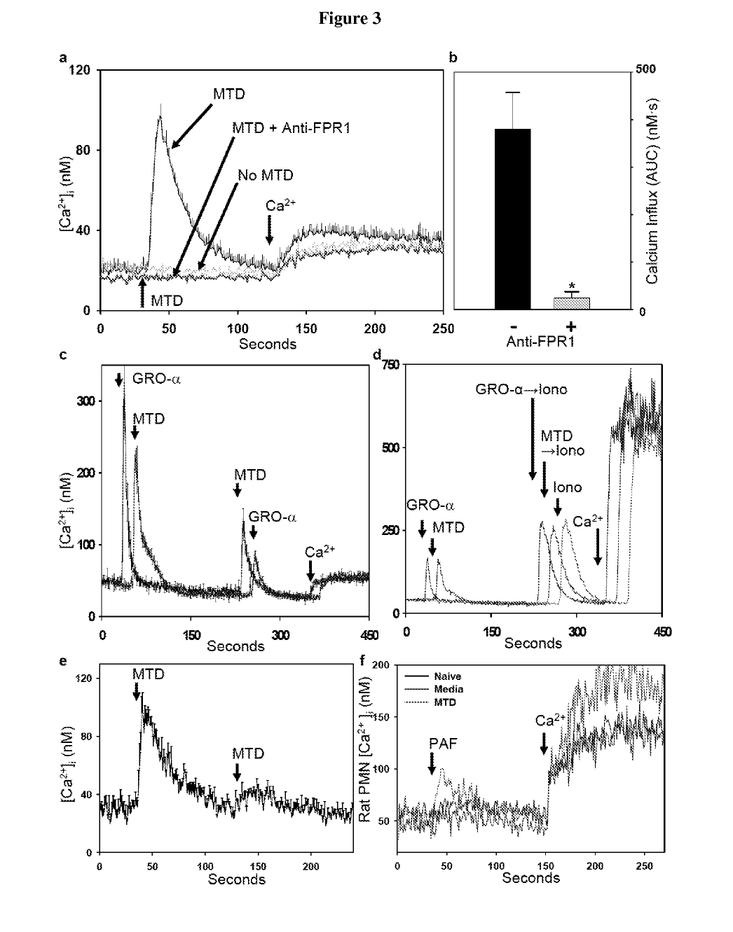

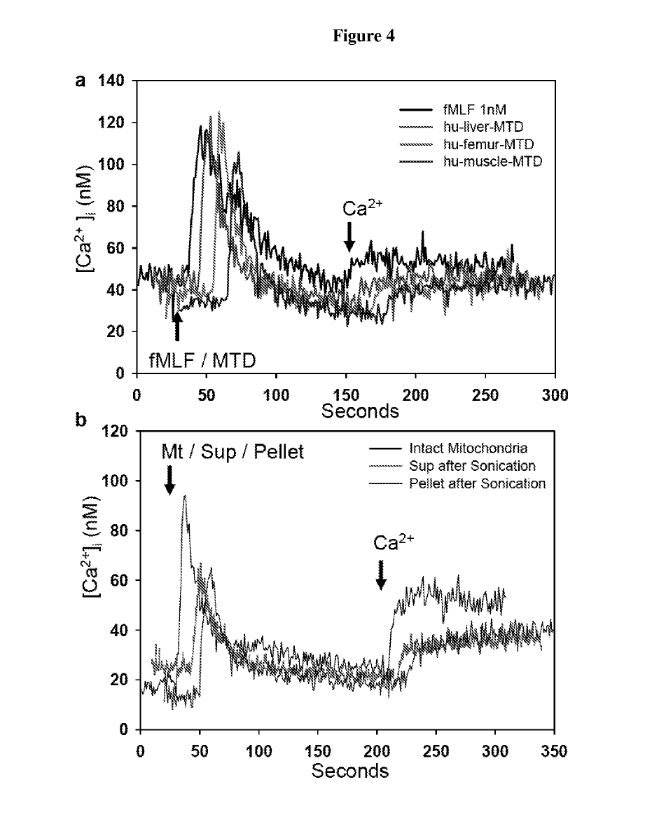

Mitochondrial DAMPs express formyl peptides, but it is unknown whether they induce either biologic or clinical inflammation. In 1975, Schiffmann et al. showed bacterial formyl peptides are PMN chemoattractants. The synthetic tripeptide N-formyl-methionyl-leucyl-phenylalanine (fMLF) has been used extensively to simulate bacterial challenge, and formyl peptides are known to signal via two G-protein coupled receptors (GPCR): FPR1 and FPRL-1, with high and low affinities, respectively. Formyl peptides can also be displayed by non-classical MHC molecules and mediate skin graft rejection. A few studies have examined the possibility that mitochondrial formyl peptides might activate PMN or related cell lines, but the role of endogenous formyl peptides in trauma, PMN activation, SIRS, and related organ failure is unstudied. PMN activation via GPCR like FPR1 typically causes increased intracellular calcium concentration ([Ca²⁺]ᵢ), heterologous and homologous GPCR desensitization, and activation of MAP kinases (MAPKs). Since elevated PMN [Ca²⁺]ᵢ is critical for inflammatory activation, the activation of PMN Ca²⁺ flux by human MTD was studied. MTD prepared from cultured human rhabdomyosarcoma cells (1.2 μg/ml protein) was found to induce human PMN [Ca²⁺]ᵢ fluxes equal to 1 nM fMLF (FIG. 3A). Multiple clinical sources of MTD (human muscle, liver, and fracture hematoma) produced similar PMN Ca²⁺ depletion responses on a protein concentration basis (FIG. 4A), as did rat muscle or liver MTD when exposed to rat or human PMN. Whole and fragmented mitochondria had very similar potency (FIG. 4B). These findings suggest that release of MTD activates immunity independent of anatomic sites of injury.

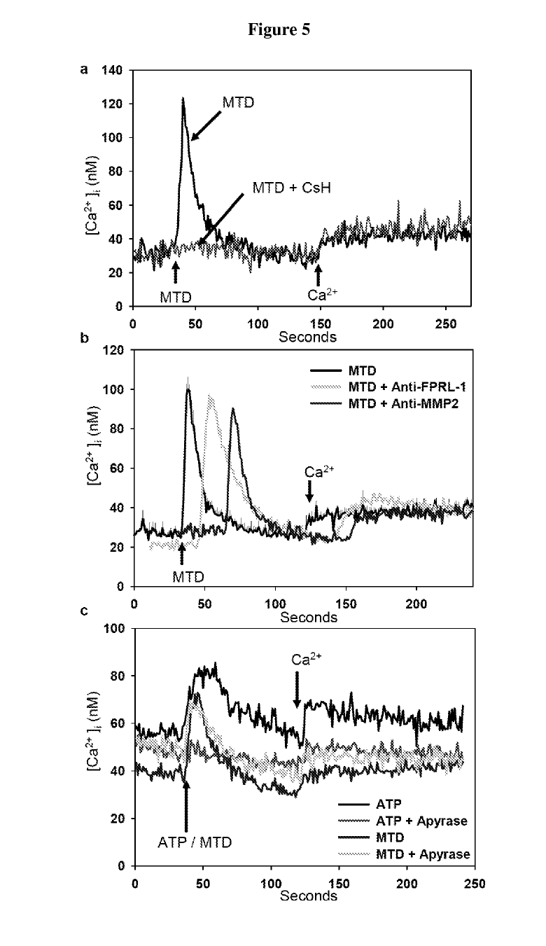

Preincubation of human PMN with monoclonal blocking antibodies for FPR1 abolished Ca²⁺ depletion (FIG. 3A) and Ca²⁺ entry (FIG. 3B) responses to MTD. The fungal metabolite Cyclosporin H (CsH, a potent inhibitor of FPR1) also abolished MTD induced Ca²⁺ flux (FIG. 5A), whereas isotype control antibodies to FPRL-1 or MMP-2 had no effects (FIG. 5b). To insure Ca²⁺ flux was not caused by ATP in MTD, MTD was treated with apyrase, which destroys ATP. Responses to apyrase-treated and untreated MTD were identical, whereas apyrase abolished [Ca²⁺]ᵢ response to 1 μM ATP (FIG. 5C). Random MTD samples were assayed for ATP (n=3) and none was detected.

| | | | Time to | | | | | | |
|---|---|---|---|---|---|---|---|---|---|
| | | Mechanism | sample | Head & Neck | | Face | | | |
| Sex | Age | of injury | (min) | injuries | AIS | injuries | AIS | Chest injuries | AIS |
| M | 60 | Motor vehicle | 128 | Scalp | 1 | | 0 | Multiple rib fractures, | 4 |
| | | crash | | hematoma | | | | flail chest, hemo- | |
| | | | | | | | | pneumothorax | |
| M | 17 | Motorcycle | 129 | Intracranial | 4 | Multiple | 2 | Pneumothorax | 5 |
| | | crash | | hemorrhage. | | facial | | Pulmonary contusions | |
| | | | | Cerebral | | fractures. | | | |
| | | | | edema. | | | | | |
| | | | | Multiple skull | | | | | |
| | | | | and facial | | | | | |
| | | | | fractures | | | | | |
| M | 41 | Fall on to | 83 | | 0 | | 0 | T12 spine fracture, rib | 4 |
| | | concrete | | | | | | fractures, bilateral | |
| | | | | | | | | hemothoraces | |
| M | 31 | Pedestrian | 76 | Intracranial | 5 | | 0 | | 0 |
| | | struck | | hemorhage. | | | | | |
| | | | | Skull fracture | | | | | |
| M | 19 | Motor vehicle | 94 | Diffuse brain | 5 | Facial | 2 | | 0 |
| | | crash | | injuries (DAI) | | fracture | | | |
| M | 59 | Fall down | 89 | Intracranial | 4 | | 0 | Bilateral pulmonary | 4 |
| | | stairs | | hemorrhage. | | | | contusions, Rib | |
| | | | | Skull fracture. | | | | fractures | |
| M | 39 | Fall down | 64 | Intracranial | 5 | | 0 | Pulmonary contusion. | 3 |
| | | stairs | | hemorrhage. | | | | Rib fractures. | |
| | | | | Skull fracture. | | | | | |
| | | | | Cerebral | | | | | |
| | | | | edema. | | | | | |
| M | 51 | Pedestrian | 93 | Intracranial | 4 | | 0 | T6, T8 spine fractures | 3 |
| | | struck | | hemorrhage. | | | | | |
| | | | | Diffuse brain | | | | | |
| | | | | injury (DAI) | | | | | |
| M | 16 | Motorcycle | 87 | Intracranial | 5 | | 0 | | 0 |
| | | crash | | hemorrhage. | | | | | |
| | | | | Diffuse brain | | | | | |
| | | | | Injury (DAI) | | | | | |
| M | 27 | Motorcycle | 142 | | 0 | Multiple | 2 | Multiple rib fractures, | 5 |
| | | crash | | | | facial | | pulmonary contusions, | |
| | | | | | | fractures | | hemopneumothorax, | |
| | | | | | | | | T3/4 spine fractures. | |
| | | | | | | | | Spinal cord injury. | |
| F | 75 | Pedestrian | 75 | Intracranial | 4 | Multiple | 0 | Rib fractures, flail chest | 4 |
| | | shuck | | hemorrhage. | | facial | | bilateral pneumo- | |
| | | | | Skull fracture. | | fractures | | thoraces and pulmonary | |
| | | | | | | | | contusions | |
| M | 71 | Motor vehicle | 111 | Cervical spine | 4 | | 0 | Rib fractures | 1 |
| | | crash | | fracture | | | | | |
| | | | | dislocations. | | | | | |
| | | | | Spinal cord | | | | | |
| | | | | injury. | | | | | |
| F | 66 | Motor vehicle | 98 | | 0 | Facial | 2 | Rib fractures, | 5 |
| | | crash- | | | | fracture | | pneumothorax | |
| | | ejected | | | | | | | |
| F | 59 | Fall 4 floors | 92 | | 0 | | 0 | | 0 |
| M | 17 | Motorcycle | 120 | Intracranial | 5 | Multiple | 3 | Pneumothorax, T6 | 3 |
| | | crash | | hemorrhage. | | facial and | | spine fracture | |
| | | | | Diffuse brain | | jaw | | | |
| | | | | injury (DAI) | | fractures | | | |

| | | | | Extremity-Pelvic | | | Systolic blood | |
|---|---|---|---|---|---|---|---|---|
| | | Abdomen-Pelvic | | girdle bony | | Global | pressure | Serum buffer base |
| Sex | Age | contents injuries | AIS | injuries | AIS | ISS | (mmHg) | Excess (U) |
| M | 60 | Lumbar spine | 2 | Pelvis and multiple | 3 | 29 | 121 | −0.8 |
| | | fracture | | arm fractures | | | | |
| M | 17 | | 0 | Wrist fracture, burn | 2 | 45 | 117 | −8.2 |
| | | | | of calf | | | | |
| M | 41 | | 0 | Pelvic and multiple | 5 | 41 | 64 | −3.6 |
| | | | | upper & lower | | | | |
| | | | | extremity fractures | | | | |
| M | 31 | | 0 | | 0 | 25 | 132 | −1.1 |
| M | 19 | | 0 | | 0 | 29 | 124 | 2.9 |
| M | 59 | | 0 | Fractured clavicle | 2 | 36 | 128 | −6.6 |
| M | 39 | | 0 | | 0 | 34 | 142 | −0.6 |
| M | 51 | Lumbar spine | 2 | Multiple lower | 3 | 34 | 71 | −10 |
| | | fracture | | extremity fractures | | | | |
| M | 16 | | 0 | | 0 | 25 | 104 | −1.2 |
| M | 27 | | 0 | Fractured scapula | 2 | 33 | 84 | −1.9 |
| F | 75 | Lumbar spine | 2 | Multiple pelvis and | 3 | 43 | 128 | −3.9 |
| | | fractures | | Upper extremity | | | | |
| | | | | fractures. | | | | |
| M | 71 | | 0 | Multiple upper | 3 | 26 | 93 | −0.3 |
| | | | | extremity fractures | | | | |
| F | 66 | | 0 | Fracture- | 2 | 33 | 134 | 3.1 |
| | | | | dislocation left hip | | | | |
| F | 59 | Abdominal | 0 | Pelvic fracture and | 5 | 25 | unmeasurable | −8.7 |
| | | hemorrhage | | retroperitoneal | | | | |
| | | | | hemorrhage | | | | |
| M | 17 | | 0 | Pelvis and lower | 2 | 43 | 144 | −3.1 |
| | | | | extremity fractures | | | | |

TABLE 2
Demographics of Volunteer Control Blood Donors.

| | | Trauma or injury | Acute | Chronic |
|---|---|---|---|---|
| Sex | Age | at time of sample | diseases | diseases |
| M | 28 | None | None | None |
| F | 62 | None | None | None |
| F | 30 | None | None | None |
| M | 56 | None | None | None |
| M | 54 | None | None | Type II |
| | | | | Diabetes |
| F | 37 | None | None | None |
| M | 61 | None | None | Type II |
| | | | | Diabetes |
| M | 32 | None | None | None |
| F | 38 | None | None | None |
| M | 33 | None | None | None |
| F | 34 | None | None | None |
| M | 26 | None | None | None |

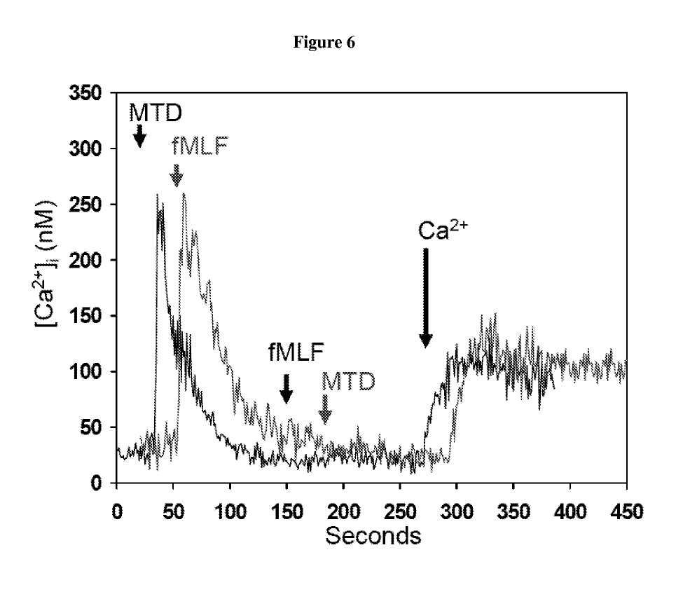

Another typical effect of FPR activation is desensitization of chemokine receptors. This is an important precursor of immune paralysis and sepsis after trauma. Human PMN were treated with MTD or the chemokine GRO-α. Sequential agonist exposure suppressed responses to the second agonist (FIG. 3C). Sequential experiments stimulating human PMN with GRO-then-ionomycin, MTD-then-ionomycin, and ionomycin alone (FIG. 3D) showed identical Ca²⁺ responses to ionomycin, which is receptor independent and reflects Ca²⁺ stores. Thus, the suppressed Ca²⁺ signaling after MTD reflected heterologous desensitization of CXCR2 by FPR1. PMN also showed complete homologous desensitization of Ca²⁺ flux when re-challenged with MTD (FIG. 3E) or fMLF (FIG. 6).

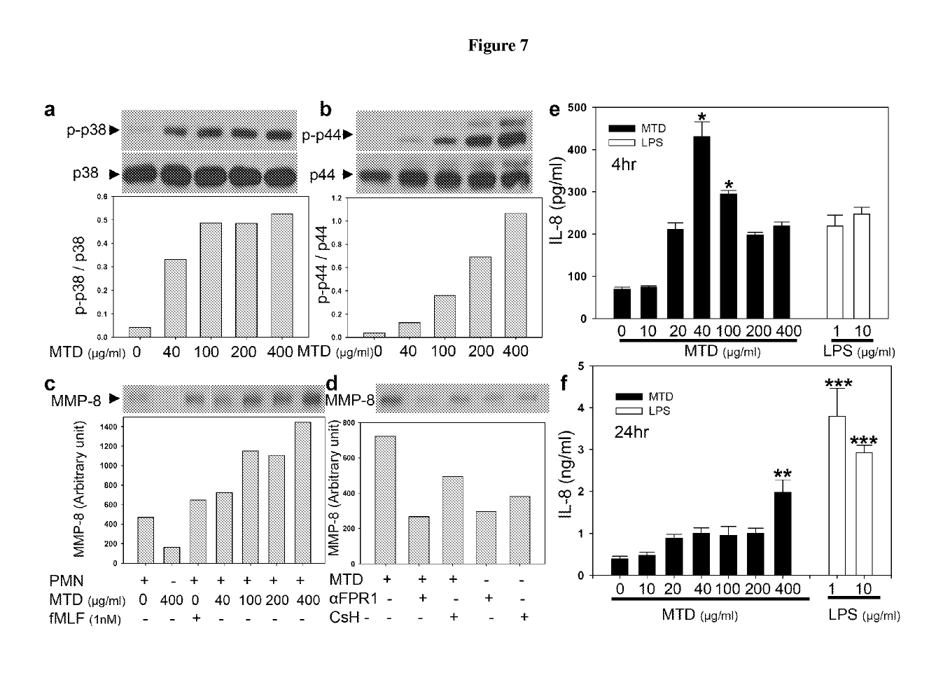

Phosphorylation of MAP kinases is another typical PMN response to injury and is necessary for activation. MTD from human skeletal muscle caused dose-dependent phosphorylation of PMN p38 and p44/42 MAP kinases (FIGS. 7A and 7B). p38 MAPK was activated at lower MTD concentrations than p44/42 MAPK. Thus, when trauma disrupts skeletal muscle-liberated mitochondrial DAMPs, these molecules can activate multiple PMN signal pathways, converting PMN to an inflammatory phenotype.

### Mitochondrial DAMPs Activate PMN Phenotype

When neutrophils are activated, they release lytic enzymes like matrix metalloproteinases (MMPs). These enzymes aid in tissue penetration and chemoattractant processing, thus favoring PMN recruitment. MMP-8 is a highly neutrophil-specific collagenase contained in secondary granules and released during inflammation. Thus, MMP-8 release reflects PMN activation and tissue MMP-8 is a marker for PMN invasion. The chemokine IL-8 is critically involved in PMN chemotaxis to lung and injured tissues. Inflammation induces PMN synthesis and release of IL-8. The data show that MTD causes MMP-8 release from human PMN: release is dose-dependent (FIG. 7C) and inhibited by either CsH or anti-FPR1, showing dependence on FPR1 (FIG. 7D). Mitochondria contain no MMP-8 (FIG. 7C). Human PMNs also synthesized and released IL-8 in response to MTD (FIGS. 7E and 7F). MTD induced more IL-8 release than LPS at 4 hours with a “bell-shaped” response curve (FIG. 7E). This may reflect homologous FPR suppression at high MTD concentration (see, FIG. 3E). At 24 hours, LPS was more potent (FIG. 7F), but MTD generally induced neutrophil responses similar in degree to LPS.

### Mitochondrial DAMPs Induce PMN Migration

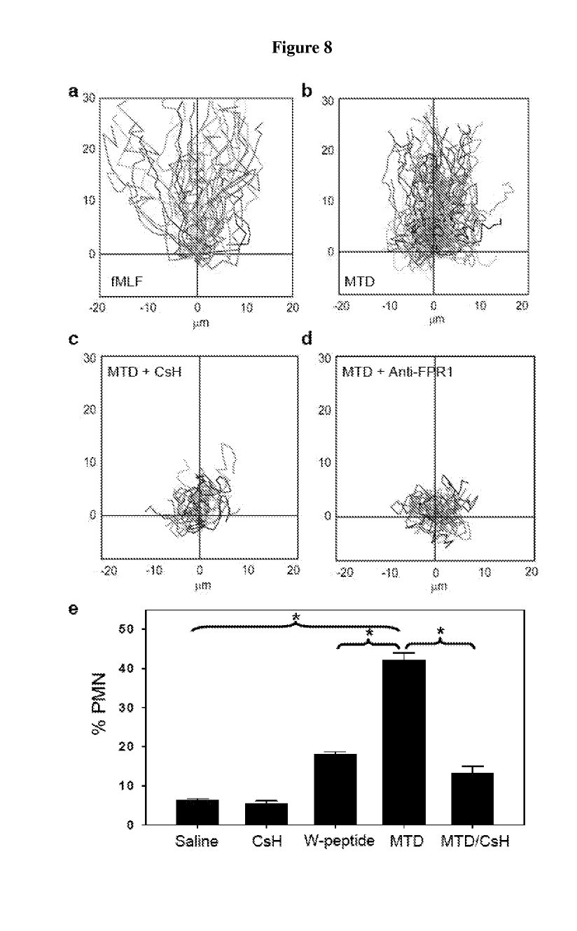

Using lytic enzymes like MMPs to penetrate barriers, PMN migrate into target organs and injure bystander tissues. The effects of MTD on human PMN migration were measured in vitro and in vivo. Using video-microscopy, PMN migration toward MTD from clinical femur fractures was examined (FIGS. 8A-8D). PMN showed brisk migration toward MTD and the speed and directionality of that migration were markedly inhibited by CsH blockade of FPR1 (FIG. 8C) or antibodies to FPR1 (FIG. 8D). These data further show that neutrophil infiltration occurred in vivo in response to clinical concentrations of MTD. This was demonstrated by placing sufficient liver-derived MTD into the peritoneum of mice to model traumatic necrosis of 10% of the mouse's liver. A rapid (2 hours) development of neutrophilic peritonitis was observed (FIG. 8E). MTD was more active than the mouse FPR agonist W-peptide (10 nM). CsH significantly reduced peritonitis, showing FPR1 dependence (FIG. 8E).

Mitochondrial DNA (mtDNA) is a Potent PMN Activator

In addition to formyl peptides, mitochondria contain their own genome. mtDNA has structural characteristics similar to bacterial DNA in that it is circular and has nonmethylated CpG motifs. Moreover, mtDNA is found in joint fluids of rheumatoid arthritis patients and induces inflammation in vivo. The data show that mtDNA is circulated after injury (FIG. 1), and CpG DNA was known to be a TLR9 agonist. However, the ability of mtDNA to activate PMN was unstudied.

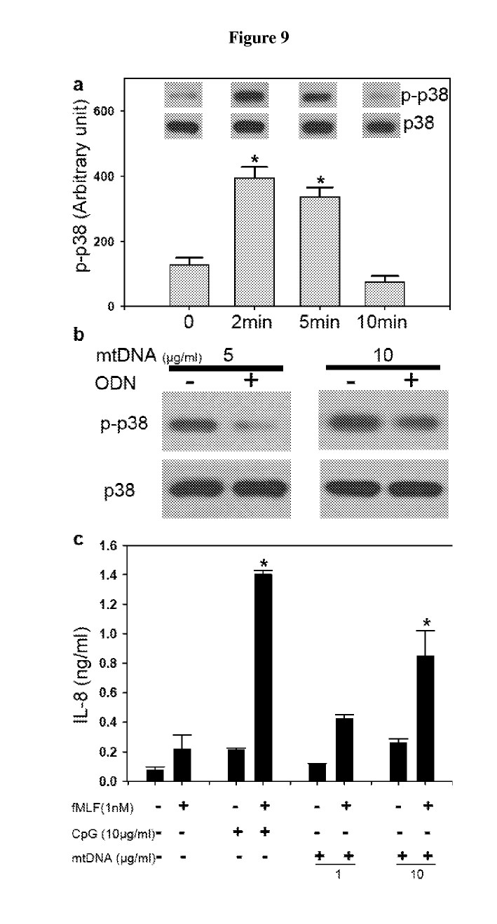

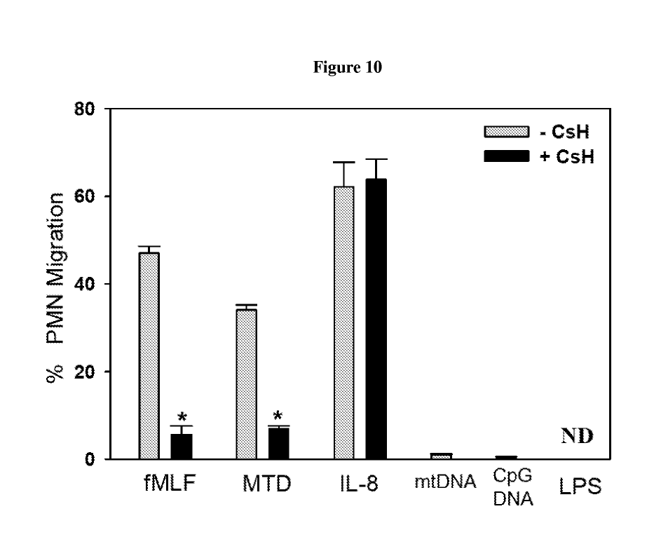

TLR9 is expressed by PMN and its engagement activates PMN p38 MAPK. Experiments were performed to determine whether PMN p38 MAPK was activated by mtDNA at the concentrations in trauma patient plasma (FIG. 1D). The data show that 1 μg/ml mtDNA caused typical p38 MAPK phosphorylation (FIG. 9A). Unlike MTD activation of FPR1 (FIG. 7A), purified mtDNA did not activate p44/42 MAPK. p38 MAPK activation was markedly diminished by the inhibitory oligodeoxynucleotide (ODN) TTAGGG (FIG. 9B). ODN binds CpG motifs, blocking interactions with TLR9. In order to study downstream signaling events, PMNs were incubated with CpG DNA (10 μg/ml) or mtDNA within the clinical range (1-10 μg/ml). CpG DNA and mtDNA released little IL-8 alone, but each promotes IL-8 release in the presence of low-dose fMLF (1 nM) (FIG. 9C). This is consistent with reports that GM-CSF primes IL-8 release mediated by CpG DNA/TLR9. Taken together, these data show that clinical mtDNA concentrations activate PMN, probably through TLR9. The TLR ligands mtDNA, CpG DNA, and LPS had no direct effect on PMN chemotaxis in transwell assays (FIG. 10).

### Mitochondrial DAMPs Cause Neutrophilic Organ Injury

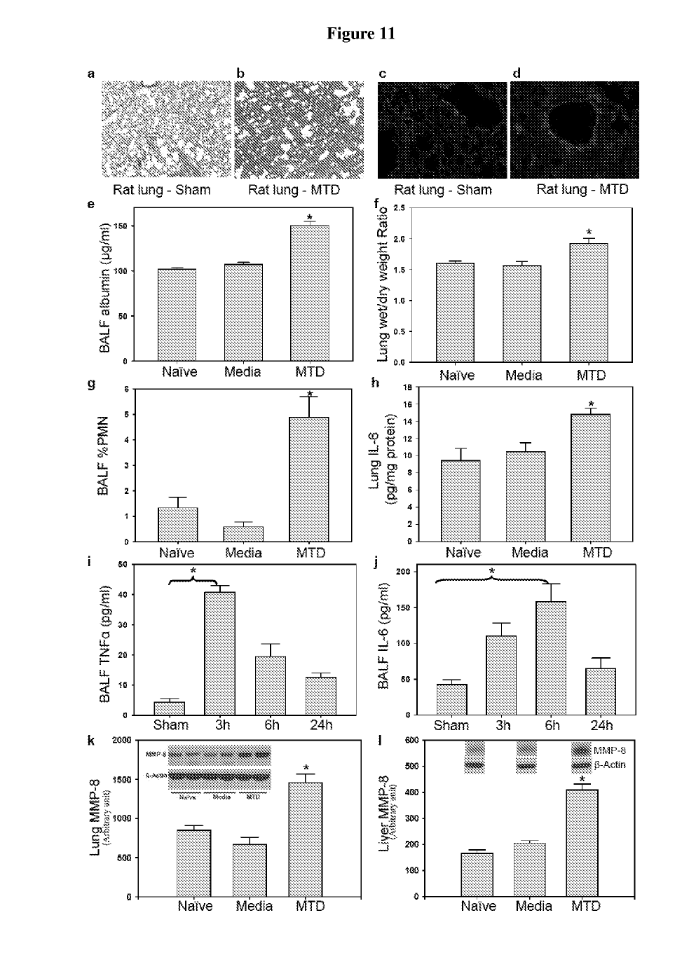

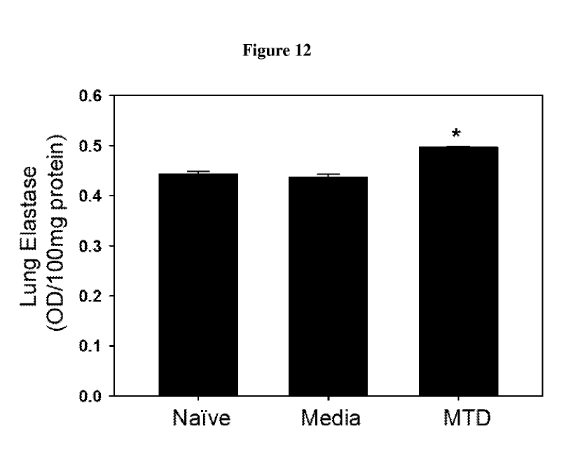

Additional experiments were performed to determine whether circulating MTD could recreate inflammatory organ injury in vivo. In these experiments, rat liver MTD equal to 5% of the rat's liver was injected intravenously. Injected animals demonstrated marked histologic inflammatory changes as early as 3 hours post-injection (FIG. 11). Free radical lung injury was documented by immunofluorescent stains for 4-hydroxy-2-nonenal (4-HNE) (FIG. 11C vs. 11D). MTD injection increased lung albumin (FIG. 11E) and wet/dry weight (FIG. 11F). MTD injection caused PMN influx into the airways (FIG. 11G), as well as accumulation of IL-6 (FIG. 11H) and elastase in lung (FIG. 12). Accumulation of MMP-8 in the lung confirmed PMN infiltration (FIG. 11K). In addition, lung inflammation correlated with an early appearance of TNF-α (FIG. 11I) and the later appearance of IL-6 (FIG. 11J) in bronchoalveolar lavages. The systemic nature of inflammation was shown by priming of circulating PMN [Ca²⁺]ᵢ mobilization (FIG. 3F) and PMN infiltration of liver as shown by MMP-8 accumulation in liver homogenates (FIG. 11L). Control rats (naïve or restrained and injected with medium) showed no evidence of inflammation in the lung or liver.

Systemic inflammation, organ injury, and immune paralysis are characteristics of major trauma as well as infection. The overlapping presentations of sterile and infective SIRS create difficult clinical management problems, and the similarity of the two syndromes can lead to presumptive diagnoses of infection and empiric antimicrobial use where other therapies could be developed. The early detection of an elevated mitochondrial DNA or protein in a sample from a patient or an increased ratio of mitochondria DNA or protein to bacterial DNA or protein in a sample from a patient may be used to identify a patient that will later develop a sterile inflammation (e.g., as a result of tissue damage).

The above data demonstrate that blunt trauma, as well as surgically-induced trauma (e.g., femur reaming) causes MTD to enter the circulation, where it is normally absent, at titers that stimulate immune cells. Thus, blunt trauma, as well as other types of tissue damage, can activate inflammation systemically independent of the anatomic site of tissue trauma. Moreover if mitochondria showed molecular similarity to their bacterial ancestors it might explain why traumatic and infective inflammation appear similar. Our data show that blunt trauma, as well as other types of tissue injury, mimic infective inflammation precisely because mitochondrial DAMPs share at least two molecular signatures with bacterial PAMPs: formyl peptides and mtDNA, each of which can act on PRRs normally recognizing bacterial PAMPs.

After major mechanical trauma (blunt trauma) MTD spills into the plasma at concentrations that activate PMN in the circulation (FIGS. 3F, 7, and 9), rather than inducing activation upon the encounter of specific targets. Such systemic PMN activation might incite non-specific organ attack (FIG. 11), while suppressing the chemotactic response to infective stimuli (FIG. 3C, 3E, and FIG. 6). Other immune cells may also be affected similarly.

We have discovered that tissue injury releases two separate classes of DAMP: mitochondrial formyl peptides and mtDNA. These molecules are likely a small subset of the DAMPs released by trauma, but they appear quite potent at the concentrations observed after injury. Nonetheless, release of other intracellular ‘alarmins’ is probably important after injury and PMN are likely not the only responders to mitochondrial DAMPs. The above data show that injury-derived DAMPs are recognized by the innate immune system and act through the same pathways as bacterial PAMPs. This discovery helps explain why responses to ancient ‘enemies within’ released by injury mimic the septic responses to invasive pathogens so closely.

### A PCR Test to Distinguish Between Sterile and Infective SIRS

In order to generate sensitive and specific probes that will distinguish between sterile SIRS associated with circulation of mitochondrial DNA from infective SIRS, the specificity and dose-response of probes for mitochondrial cytochrome-B and bacterial 16S ribosomal RNA to mtDNA from human liver and bacterial DNA (bDNA) from the 3 major clinical groups of infective organisms (Gram(+), Gram(−), and anaerobic bacteria) was studied. Genomic bacterial DNAs from S. aureus, E. coli, and B. fragilis were tested at concentrations of 1 fg/μL, 1 pg/μL, and 1 ng/μL. mtDNA prepared from human liver was tested at a concentration of 1 pg/μL, 1 ng/μL, and 1 μg/μL.

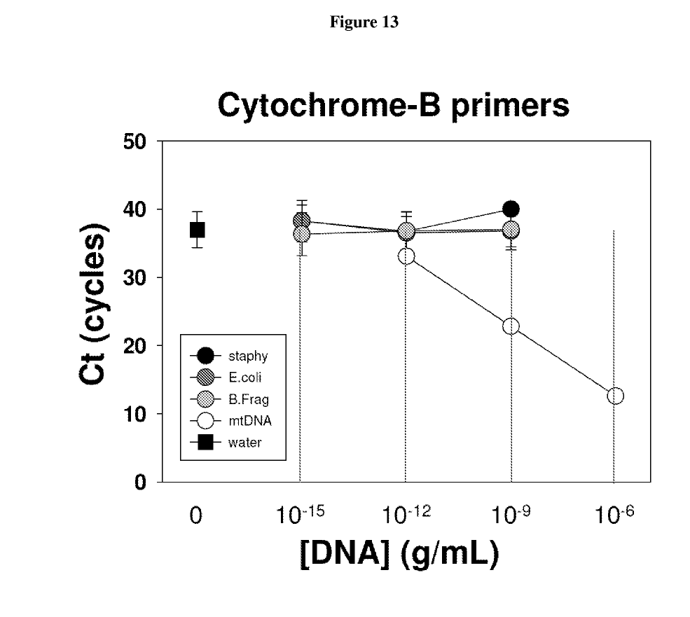

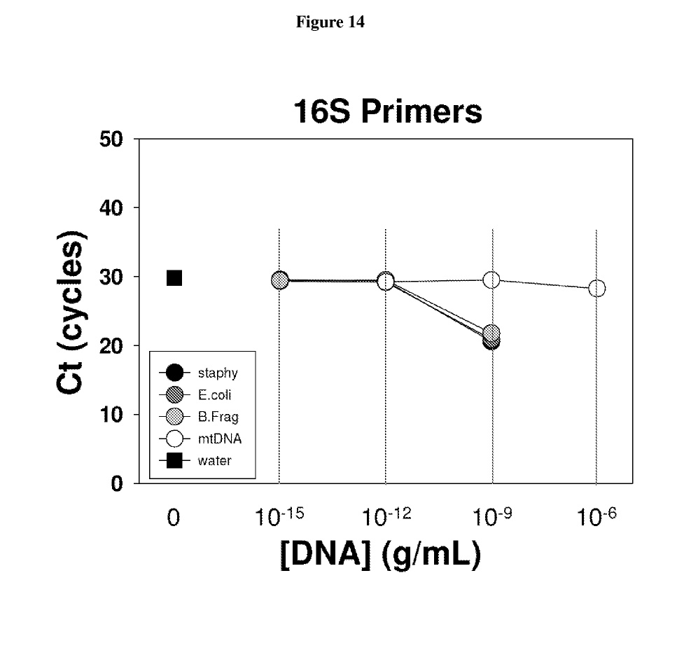

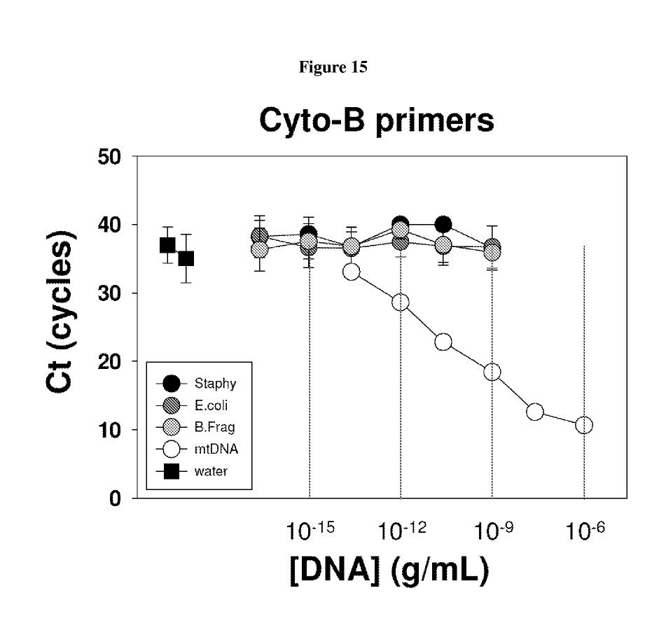

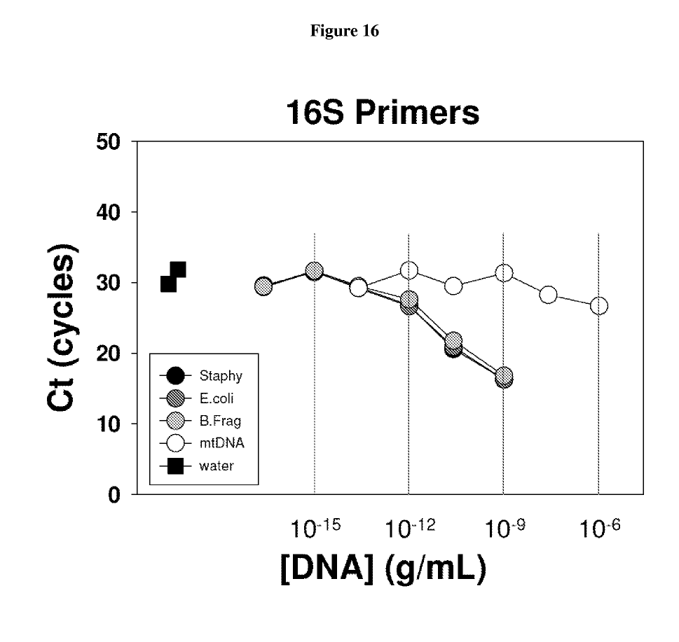

The results of these tests are shown in FIGS. 13-16. The cytochrome-B primers detected mtDNA at very low concentration, whereas they failed to detect bDNA even at high concentration (FIG. 13). Furthermore, the primers for 16S bacterial ribosomal RNA (16S rRNA) detect all bacterial phyla identically (FIG. 14). Moreover, the cytochrome-B primers detected mtDNA in a linear fashion across 9 log units of concentration, whereas they showed no response to bacterial DNA (identical to water; FIG. 15). Higher concentrations were not tested, since those tested were already higher than clinical concentrations. The probes for 16S rRNA became detectable at pg/ml concentrations. Detection was universal and was identical across the bacterial phyla (FIG. 16).

These data show that our Cyto-B and 16S rRNA primers specifically recognize samples of mitochondrial DNA and bacterial DNA respectively. All three clinically important groups of bacterial (Gram (+), Gram(−), and anaerobes) are recognized identically by the 16S rRNA primers. The threshold mtDNA concentration required for recognition of Cyto-B was in the femtogram/mL range. Bacterial DNA was recognized by our 16S rRNA primers in the picogram/mL range. The Cyto-B primers showed no cross-reaction with 1 ng/mL of bacterial DNA. Finally, the 16S rRNA primers showed no cross-reaction with mtDNA at up to 1 μg/mL of mitochondrial DNA. Testing based on these primers should allow for clinical discrimination between infective and sterile SIRS and the prediction of the likelihood of a subject to later develop a sterile inflammation (e.g., a sterile systemic inflammation).

In practice, a clinical test for infective vs. sterile inflammation or the identification of a subject having an increased likelihood to later develop a sterile inflammation may be performed by measuring the amount of bacterial DNA/peptide, measuring the amount of mitochondrial DNA/peptide, and determining the ratio therebetween to determine a confidence level of whether a patient has infective or sterile inflammation or has an increased likelihood of later developing a sterile inflammation (e.g., a sterile systemic inflammation). Confidence intervals can be established prior to testing using empirical data. For example, based on empirical data, it may be found that 50% of patients having a bacterial/mitochondrial DNA ratio less than 1 have infective inflammation. A series of such empirical confidence intervals can be established for different ratios, and the patient's test result can then be matched to these confidence intervals to give an outcome. In some cases, it may be that both infective and sterile inflammation are present simultaneously. In these cases, the absolute amounts of DNA/peptide will be informative.

### Methods

Compliance, Human and Animal Experimentation

Collection of human specimens and samples was approved by the internal review board. Care of experimental animals was in accordance with NIH guidelines and approved by the internal review board.

Isolation of Mitochondria, Preparation of Mitochondrial DAMPs Suspension (MTD), and Mitochondrial DNA (mtDNA)

Mitochondria were isolated from rat liver or muscle, and from human rhabdomyosarcoma cells, liver, skeletal muscle, or femur fracture reaming specimens. Clinical liver injury, muscle crush injury, and femur fracture fixation by reamed nailing are all common, important events closely linked to inflammation and acute lung injury after injury. Clinical samples used to prepare mitochondria were harvested from patients receiving antibiotics. MTD and mtDNA were prepared under sterile conditions. Endotoxin levels were measured by limulus amebocyte lysate assay and did not achieve significant levels.

### Real-Time PCR

The primers used in real-time PCR (Table 3) were synthesized by Invitrogen (Carlsbad, Calif.).

| | | (SEQ |
|---|---|---|
| | | ID |
| Gene | Sequence | NO:) |
| Human-cytochrome B | 5′-atgaccccaatacgcaaaat-3′ (forward) | (1) |
| | 5′-cgaagtttcatcatgcggag-3′ (reverse) | (2) |
| Human-cytochrome C | 5′-atgaccccaatacgcaaaat-3′ (forward) | (3) |
| oxidase subunit III | 5′-cgaagtttcatcatgcggag-3′ (reverse) | (4) |
| Human-NADH dehydrogenase | 5′-atacccatggccaacctcct-3′ (forward) | (5) |
| | 5′-gggcctttgcgtagttgtat-3′ (reverse) | (6) |
| Rat-cytochrome B | 5′-tccacttcatcctcccattc-3′ (forward) | (7) |
| | 5′-ctgcgtcggagtttaatcct-3′ (reverse) | (8) |
| Rat-cytochrome C | 5′-acataccaaggccaccaac-3′ (forward) | (9) |
| oxidase subunit III | 5′-cagaaaaatccggcaaagaa-3′ (reverse) | (10) |
| Rat-NADH dehydrogenase | 5′-caataccccacccccttatc-3′ (forward) | (11) |
| | 5′-gaggctcatcccgatcatag-3′ (reverse) | (12) |
| Bacterial 16S ribosomal | 5′-cgtcagctcgtgttgtgaaa-3′ (forward) | (13) |
| RNA | 5′-ggcagtctccttgagttcc-3′ (reverse) | (14) |

PMN Studies—General

PMN isolation (Hauser et al., J. Leukoc. Biol. 69:63-68, 2001; Fekete et al., Shock 16:15-20, 2001), cytosolic calcium studies (Hauer et al., J. Leukoc. Biol. 69:63-68, 2001; Hauser et al., J. Trauma 48:592-598, 2000; Tarlowe et al., J. Immunol. 171:2066-2073, 2003; Grynkiewicz et al., J. Biol. Chem. 260:2440-2450, 1985), IL-8 synthesis measurement by ELISA (Zallen et al., J. Trauma 46:42-48, 1999), cell signaling by Western blots (Zhang et al., Life Sci. 77:3068-3077, 2005), transwell chemotaxis (Tarlowe et al., J. Immunol. 171:2066-2073, 2003), and video-microscopy chemotaxis assays (Chen et al., Science 314:1792-1795, 2006) were performed as previously described.

Rat Administration of MTD

Male Sprague-Dawley rats (300-350 g, Charles River, Wilmington, Mass.) were treated with intravenous MTD. Blood volume was estimated from weight (Hauser et al., Shock 24 (Suppl). 1:24-32, 2005). qPCR of plasma from injected rats showed mtDNA levels of 122±22 ng/ml 1 hour after injection (nl<<1 ng/ml). Leukocytes in bronchoalveolar lavage fluids (BALF) were counted by hemocytometer. PMN were counted by cytospin. For pathology, the lungs were inflated gently and immersed in formalin prior to stain with hematoxylin and eosin or immunohistochemical stain for 4-HNE.

Reagents and Chemicals

fMLF, ethyleneglycol-bis(β-aminoethylether)-N,N′-tetraacetic acid (EGTA), protease inhibitor cocktail, and DMSO were purchased from Sigma (St Louis, Mo.). Fura-2 AM, Calcein AM, and digitonin were purchased from Molecular Probes (Eugene, Oreg.). Anti-human FPR1, anti-human FPRL-1, anti-human MMP-2, anti-human MMP-8, and anti-rat MMP-8 antibodies were purchased from R&D (Minneapolis, Minn.). Antibodies to phospho-p38 MAPK (Thr180/Tyr182), p38 MAPK, phospho-p44/42 MAPK (Thr202/Tyr204), and p44/42 MAPK were from Cell Signaling (Danvers, Mass.). Donkey anti-goat IgG-HRP was obtained from Santa Cruz (Santa Cruz, Calif.). ImmunoPure Goat Anti-Rabbit IgG (peroxidase conjugated) was purchased from Pierce Biotechnology (Rockford, Ill.). Cyclosporin H (CsH) was obtained from LKT Laboratories (St Paul, Minn.). ATP bioluminescence assay kits were purchased from Roche (Palo Alto, Calif.). W-peptide was purchased from Phoenix Pharmaceuticals (Burlingame, Calif.). CpG DNA was obtained from Cell Sciences (Canton, Mass.). ODN TTAGGG was purchased from InvivoGen (San Diego, Calif.).

Mitochondrial Isolation from Clinical Material

The Mitochondria Isolation Kit for Tissue (Pierce, Rockford, Ill.) was used to isolate mitochondria from rat liver, human skeletal muscle (pathologic specimens amputated due to vascular disease), human femur medullary reamings from patients undergoing repair of femur fractures, and human liver from the margins of hepatic tumor resections. The Mitochondrial Isolation Kit for Cultured Cells (Pierce, Rockford, Ill.) was used to isolate mitochondria from human rhabdomyosarcoma cells (ATCC, Manassas, Va.). Mitochondria were isolated under sterile conditions at 4° C.

Preparation of Mitochondrial DAMPs (MTD) and mtDNA

Isolated mitochondrial pellets from tissue specimens (200 mg) or rhabdomyosarcoma cells (6×10⁷ cells) were suspended in 1 mL of HBSS. Protease inhibitor cocktail (1:100) was added to the suspension. The detection of significant amounts of circulating mtDNA in trauma patients indicated that mechanical tissue injury and/or tissue necrosis was disrupting mitochondria to some extent in vivo. The experimental preparations were standardized with routine sonication on ice (VCX130-Vibra Cell, Sonics and Materials, Newtown, Conn.) at 100% amplitude (10×, 30 s each time with 30 s intervals). The disrupted mitochondrial suspensions were then centrifuged at 12,000 rpm for 10 minutes at 4° C., followed by 100,000g at 4° C. for 30 minutes. Residual supernatants were used for experiments. Protein concentrations of the MTD solutions were determined by BCA assay (Pierce, Rockford, Ill.). mtDNA was extracted from the isolated mitochondria of various tissues using DNeasy Blood & Tissue kit (Qiagen, Valencia, Calif.). mtDNA concentration was determined spectrophotometrically. No protein contamination was found and nuclear DNA was less than 0.01% by qPCR.

Real Time PCR Protocols

Plasma DNA was prepared by QIAamp UltraSens Virus kit (Qiagen, Valencia, Calif.). Real time PCR standard curves were created to quantify mtDNA concentration by using purified mtDNA and Cytochrome B as targets. Samples that produced no PCR products after 40 cycles were considered “undetectable” and Ct (threshold) set to 40 for statistical purposes.

PMN Isolation

Detailed protocols for PMN isolation are published elsewhere (Hauser et al., J. Leukoc. Biol. 69:63-68, 2001; Fekete et a1., Shock 16:15-20, 2001). Hypotonic lysis was performed on ice to remove contaminating RBC. This method results in preparations containing ≧98% neutrophils as confirmed by flow cytometry with monocytes ˜0.02%. Viability was ≧98% as assessed by Trypan Blue.

Chemotaxis Assays by Fluorescence Videomicroscopy

Time-lapse video-microscopic chemotaxis was assayed as described previously (Chen et al., Science 314:1792-1795, 2006). Cells were exposed to a chemoattractant gradient field by slowly releasing MTD (˜100 μg/ml) or fMLF (10 nM) from a micropipette tip placed in proximity to the cells. The migration paths of individual cells were plotted using Adobe Illustrator. Cells were pretreated with or without 1 μM CsH for 5 minutes or 12.5 μg/ml anti-human-FPR1 for 10 minutes. Experiments were repeated with multiple PMN and MTD isolates. Specificity of CsH for FPR1 was examined in transwell chemotaxis assays. CsH significantly inhibited fMLF and MTD chemotaxis with no effects on IL-8.

In vivo Chemotaxis

Male mice (8-10 week, Charles River, Wilmington, Mass.) were used in this study. Mice were lightly anesthetized by isoflurane inhalation. CsH (10 μM) or DMSO was injected intraperitoneally (i.p.). After 30 minutes, 1 mL of saline or W-peptide (10 nM) or MTD (100 μg/mL, equal to the mitochondria released by a 10% liver injury) was injected i.p. Two hours later a peritoneal lavage was performed and collected for total and differential cell counts. Cell counts were performed on cytospin preparations stained with HEMA 3 (Fisher Scientific, Kalamazoo, Mich.).

PMN Degranulation Assays

PMN degranulation was assessed by measuring MMP-8 release. Human PMNs were suspended in HBSS with 1.8 mM Ca²⁺ at 37° C. for 10 minutes while exposed to MTD, mtDNA, or fMLF at indicated concentrations. For inhibitor studies, PMN were pretreated with CsH (10 μM, 5 minutes at 37° C.), anti-FPR1 antibody (12.5 μg/ml, 10 minutes at 37° C.), or control antibodies (as noted), or Inhibitory ODN TTAGGG (10/1 inhibitor to stimulus). After stimulation, PMN were placed on ice and centrifuged. Supernatants were then assayed for MMP-8 by Western blot. Residual PMN were lysed to assay for MAPKs.

ELISA

PMN were treated with various agonists for 4 hours or 24 hours (Zallen et al., J. Trauma 46:42-48, 1999). IL-8 was measured by human CXCL8/IL-8 (R&D, Minneapolis, Minn.). Experiments were performed in triplicate. TNF-α and IL-6 in rat BALF or lung were measured using BD OptEIA™ rat TNF and IL-6 ELISA sets (BD, San Diego, Calif.). Airway albumin was measured by rat albumin ELISA Quantitation kit (BETHYL, Montgomery, Tex.).

Statistical Analysis

Study data was assessed for statistical significance using Student's (unpaired) t-test or Analysis of Variance (ANOVA) where appropriate using a SigmaStat program with post-hoc tests chosen by the computer. [Ca²⁺]ᵢ transients are reported as the mean change from basal [Ca²⁺]ᵢ in nanomoles per liter (nM/L). Prolonged [Ca²⁺]ᵢ fluxes are reported as the area under the curve (AUC) of measured change from basal [Ca²⁺]ᵢ over the observation period (nM·sec). All data are reported as mean±s.e.m. with significance accepted at p<0.05.

### Example 2

### Clinical Trauma Releases Mitochondrial DAMPs into the Circulation

Trauma/Hemorrhagic Shock Releases mtDNA into the Circulation

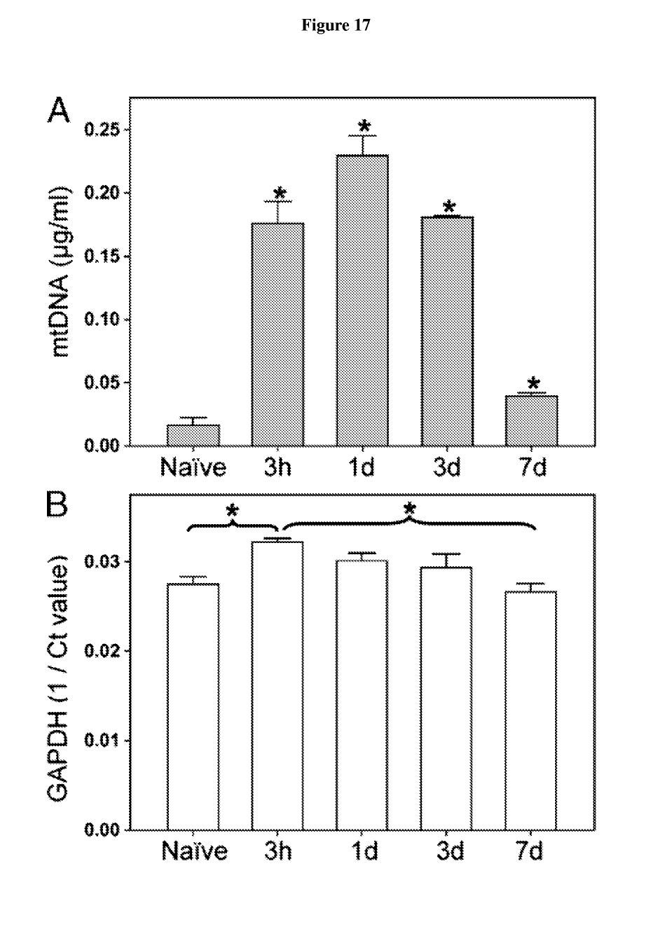

Experiments were performed to determine whether mtDNA was released into the circulation in response to trauma/hemorrhagic shock (T/HS) in a rat model. Real-time PCR was used to evaluate mtDNA and nuclear DNA (nDNA) in the plasma of naive rats and of rats subject to T/HS. As seen in FIG. 17A, there was a marked elevation in plasma mtDNA levels in rats subjected to T/HS as compared with naive rats (p<0.001). mtDNA levels reached a peak at 1 day and gradually declined thereafter, but levels were still significantly elevated seven days after T/HS. Glyceraldehyde 3-phosphate dehydrogenase (GAPDH), a genomic DNA marker, was only significantly increased 3 hours after T/HS (FIG. 17B). These data indicate that T/HS causes tissue damage, which releases both mtDNA and nDNA into the circulation.

mtDNA Activates PMN p38 MAPK

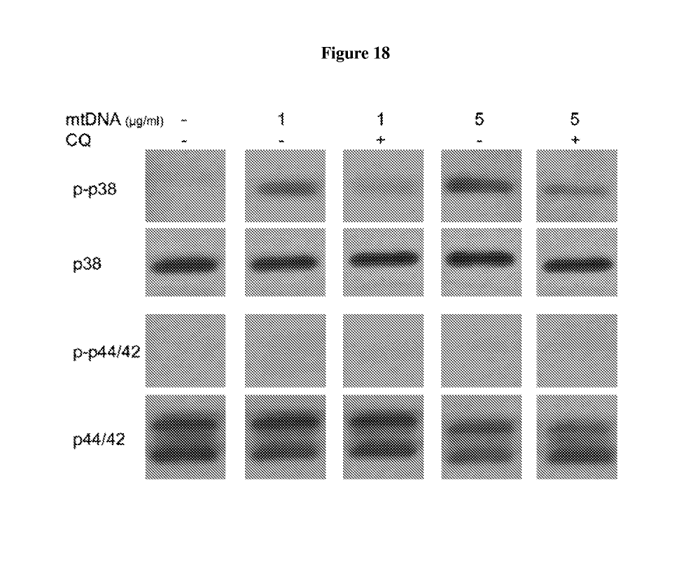

Additional experiments were performed to determine whether either mtDNA or nDNA stimulates immune cells. In these experiments, the responses of PMN to mtDNA and nDNA were measured in vitro. MAPKs are key early intermediaries in PMN inflammation. Moreover, they are downstream targets of TLR signaling pathways. To characterize the intracellular signalling mechanisms stimulated by mtDNA and nDNA, the phosphorylation of p38 and p44/42 MAPK was examined in PMN stimulated by DNA. These data show that mtDNA induced p38, but not p44/42 MAPK activation (FIG. 18).

mtDNA may Activate PAM Release of MMPs

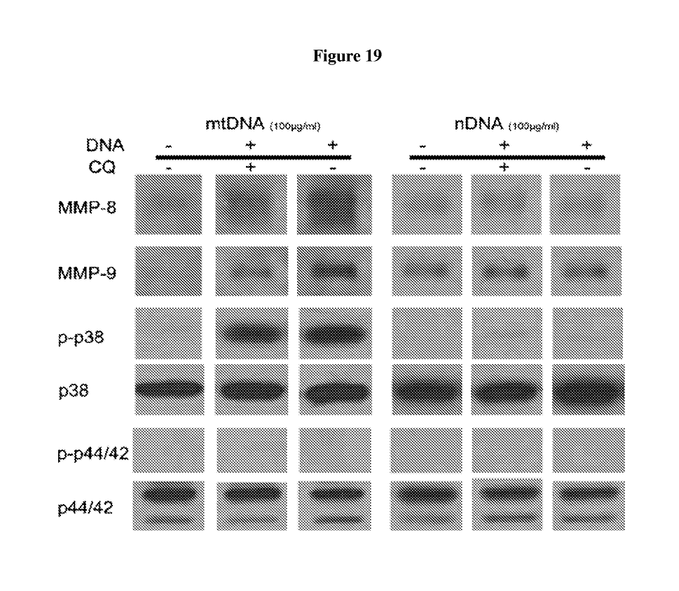

The release of matrix metalloproteases (MMPs) is crucial to many PMN inflammatory functions. The degranulation of MMPs was measured in order to further study the effect of mtDNA on PMN inflammation. PMNs from healthy volunteers were incubated with either mtDNA or nDNA (each 100 μg/mL for 30 minutes). These data show that mtDNA, at this high concentration, stimulates PMN to release MMP-8 and -9 (FIG. 19, left panel). No release of MMPs was seen at lower concentrations. Preincubation with chloroquine suppressed release of both MMP-8 and -9; however, no p38 MAPK inhibition was observed at the high mtDNA 20 concentrations needed to achieve MMP release.

Mitochondrial Debris (MTD) Causes Hepatic Inflammation in vivo

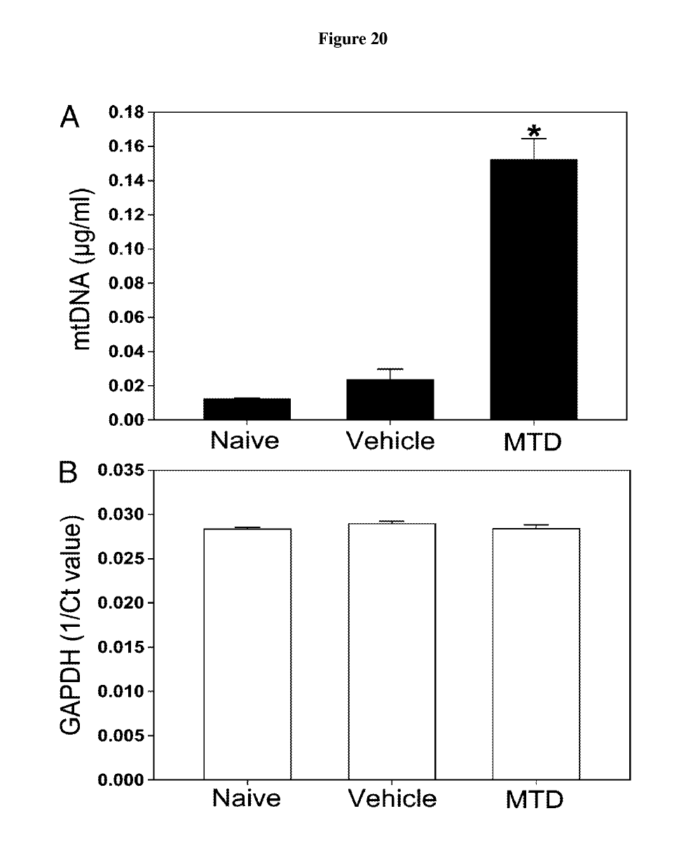

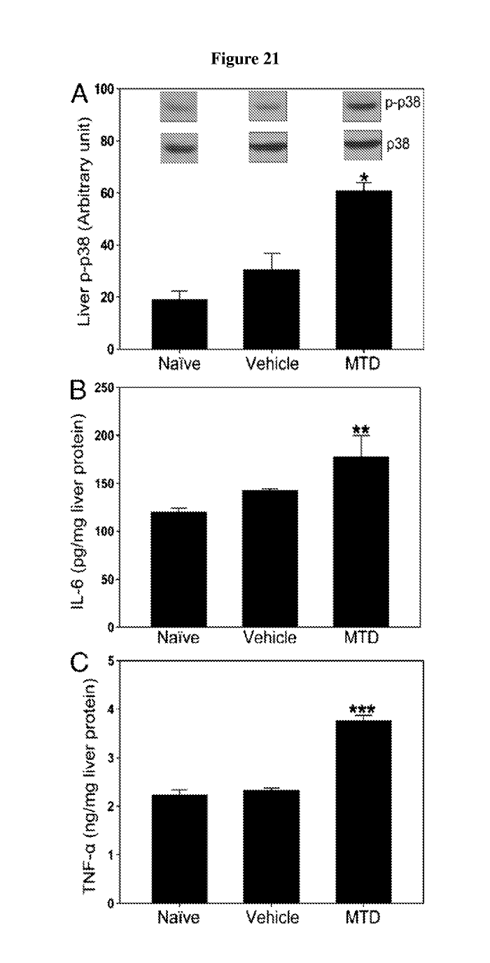

The above data demonstrate that mtDNA circulates after T/HS and activates PMN in vitro. Rats were injected with preparations of mitochondrial debris (MTD) containing 3 μg/μL mtDNA (assay by qPCR) in order to assess whether circulation of mitochondrial breakdown products might participate in the causation of inflammatory organ injury in vivo. As shown in FIG. 20A, the plasma mtDNA concentration was significantly elevated in the tail vein injection group at 1 hour, but there was no elevation found of the nuclear DNA marker GAPDH (FIG. 20B). In examining the liver for inflammation due to MTD injection, the data demonstrate that MTD caused increased phosphorylation of p38 MAPK (FIG. 21A), but not activation of p44/42 MAPK (data not shown) on assay of whole liver homogenates. Liver inflammatory responses, assessed by measurement of IL-6 (FIG. 21B) and TNF-α (FIG. 21C), were found to be elevated in the liver 1 hour after MTD injection.

### Methods

Compliance, Human, and Animal Experimentation

Collection of human specimens and peripheral blood samples was reviewed and approved by the internal review board. Care of experimental animals was in accordance with NIH guidelines and approved by the internal review board.

Preparation of Mitochondrial Debris (MTD) and mtDNA

Mitochondria were isolated from rat liver acquired from Male Sprague-Dawley rats (300-350 g, Charles River) or from human liver tissue obtained at the uninvolved margins of hepatic tumor resections. Mitochondrial isolation kits for tissue (Pierce, Rockford, Ill.) were used to isolate liver mitochondria according to the protocol for Dounce homogenization of soft tissue supplied by the manufacturer. Mitochondria were isolated under sterile conditions at 4° C.

Mitochondrial pellets from rat or human hepatocytes were isolated and suspended in HBSS buffer containing 140 mM NaCl, 5 mM KCl, 1 mM MgCl₂, 1 mM CaCl₂, 10 mM glucose, 20 mM N-2-hydroxyethylpiperazine-N′-2-ethanesulfonic acid (HEPES). The nuclear fraction of the hepatocytes was reserved for preparation of nDNA by identical methods.

To prepare a suspension of disrupted mitochondria (MTD), a protease inhibitor cocktail (1:100) was added to the suspension, which was then subjected to sonication on ice using a VCX130-Vibra Cell (Sonics and Materials, Newtown, Conn.) at 100% amplitude, 10 times for 30 seconds each with 30 seconds interval. The disrupted mitochondrial suspensions were then centrifuged at 15,000 g for 10 minutes at 4° C. followed by 100,000 g at 4° C. for 30 minutes. Supernatants containing soluble MTD were removed and stored at −80° C. for experiments. mtDNA concentration of the MTD solutions was determined by qPCR. Protein concentrations of the MTD solutions were determined by BCA assay (Pierce, Rockford, Ill.).

mtDNA and nDNA were extracted from the isolated mitochondrial pellets or nuclear fractions using DNAeasy Blood & Tissue kits (Qiagen, Valencia, Calif.) according to the manufacturer's protocol. mtDNA and nDNA concentrations and purity were determined by spectrophotometry. To insure the purity of mtDNA and to exclude contamination by nDNA, real-time PCR was used to probe samples for the presence of the mitochondrial genes cytochrome B (Cyt B), cytochrome c oxidase subunit III (COX III), and NADH dehydrogenase (NADH), as well as for the presence of the nuclear DNA marker GAPDH. In DNA prepared from mitochondria, GAPDH was at the limits of detection in the sample and nDNA was <0.1% of the total on qPCR of mtDNA samples. Thus, the mtDNA used in subsequent experiments was essentially free of nDNA. In addition, the protein contents of the mtDNA samples were examined by spectrophotometry. The A260/280 ratio was 1.8-2.0, excluding any significant contamination by protein. This was further confirmed by BCA assay and SDS-PAGE electrophoresis with Coomassie staining.

Real-Time PCR Protocols

DNA was prepared from 200 μL plasma using QIAamp DNA Blood Mini kit from Qiagen (Valencia, Calif.) according to the manufacturer's protocol. The same amount of DNA was used for each Real Time PCR reaction using SYBR Green Master Mix (Applied Biosystems, Foster City, Calif.) by Mastercycler ep realplex from Eppendorf (Foster City, Calif.). Primers for mtDNA markers Cyt B, COX III, NADH, and the nuclear DNA marker, GAPDH, were synthesized by Invitrogen (Table 4). A standard curve was created to quantify mtDNA concentration using purified mtDNA with Cyt B as the target. All data analysis was done according to the manufacturer's protocol.

### PMN Isolation

Detailed protocols for PMN isolation are published in Hauser et al. (J. Leukoc. Biol. 69:63-68, 2001). Briefly, human whole blood from healthy volunteers was collected in heparinized (10 U/mL) tubes by venipuncture and centrifuged for 10 minutes at 200 g. Platelet-rich plasma was removed. The buffy coat and red blood cells (RBC) were then centrifuged at 460 g for 30 minutes after layering onto 5 mL Polymorphoprep gradient solution (Axis-Shield PoC AS, Oslo, Norway). Supernatants containing the mononuclear cell layers were aspirated and discarded. The neutrophil layer was then collected, diluted with an equal volume of 0.45% NaCl solution, and allowed to rest for 6 minutes at room temperature in order to restore normal cellular osmolarity. PMN were then washed with RPMI 1:10 (Invitrogen,

TABLE 4
Real Time PCR Primers

| | | (SEQ |
|---|---|---|
| | | ID |
| Gene | Sequence | NO:) |
| Human cytochrome B (CytB) | 5′-atgaccccaatacgcaaaat-3′ (forward | (1) |
| | 5′-cgaagtttcatcatgcggag-3′ (reverse) | (2) |
| Human cytochrome C oxidase | 5′-atgacccaccaatcacatgc-3′ (forward) | (15) |
| subunit III (COX III) | 5′-atcacatggctaggccggag-3′ (reverse | (16) |
| Human NADH dehydrogenase | 5′-atacccatggccaacctcct-3′ (forward) | (5) |
| (NADH) | 5′-gggcctttgcgtagttgtat-3′ (reverse) | (6) |
| Human GAPDH | 5′-agggccctgacaactctttt-3′ (forward) | (17) |
| | 5′-ttactccttggaggccatgt-3′ (reverse) | (18) |
| Rat cytochrome B | 5′-tccacttcatcctcccattc-3′ (forward) | (7) |
| | 5′-ctgcgtcggagtttaatcct-3′ (reverse) | (8) |
| Rat cytochrome C oxidase | 5′-acataccaaggccaccaac-3′ (forward) | (9) |
| subunit III | 5′-cagaaaaatccggcaaagaa-3′ (revers | (10) |
| Rat NADH dehydrogenase | 5′-caataccccacccccttatc-3′ (forward) | (11) |
| | 5′-gaggctcatcccgatcatag-3′ (reverse) | (12) |
| Rat GAPDH | 5′-gaaatcccctggagctctgt-3′ (forward) | (19) |
| | 5′-ctggcaccagatgaaatgtg-3′ (reverse) | (20) |

Eugene, Oreg.), and centrifuged at 200 g for 10 minutes. A brief (30 second) hypotonic lysis is performed on ice to remove contaminating RBC. This method results in a preparation containing ˜98% neutrophils by stain or flow cytometry with a viability of ≧99% as assessed by Tyrpan Blue exclusion.

PMN Activation Assays

PMN activation was assessed by measuring MMP-8/9 degranulation and intracellular MAPK phosphorylation. Human PMN were exposed to mtDNA and nDNA at 1-100 μg/mL, 30 minutes at 37° C. After stimulation, PMN were placed on ice and centrifuged (10,000 g at 4° C. for 2 minutes) to obtain supernatants, which were then assayed for MMP-8 and MMP-9 by Western blot as described below. M-PER (Pierce, Rockford, Ill.) was then used to lyse the PMN cell pellets for determination of total and phosphorylated MAPKs by Western blot as described below. Total protein concentration of the lysates was determined by BCA assay.

Western Blotting

Rat liver homogenates and supernatants from human PMN or human PMN lysates were boiled for 5 minutes in SDS sample buffer. Proteins were separated by SDS-PAGE using a 2-40% polyacrylamide gel. Tris-glycine polyacrylamide gradient gels were purchased from Novex (San Diego, Calif.). Separated proteins were transferred to nitrocellulose membrane (0.45 μm pore size; Bio-Rad, Hercules, Calif.). Anti-MMP-8 was purchased from R&D (Minneapolis, Minn.). Antibody to MMP-9 was purchased from Santa Cruz (Santa Cruz, Calif.). Antibodies to phospho-p38 MAPK (Thr180/Tyr182), total p38 MAPK, phospho-p44/42 MAPK (Thr202/Tyr204), and total p44/42 MAPK were obtained from Cell Signaling (Danvers, Mass.). Densitometry was performed on scanned Western blot films using the Scion Image program (Scion Corp, Frederick, Md.).

Animal Model of Trauma/Hemorrhagic Shock

The model of rat T/HS used is described in Lee et al. (Shock 30:29-35, 2005). Briefly, male Sprague-Dawley rats (300-350 g, Charles River) were anesthetized with 50 mg/kg pentobarbital sodium administered intraperitoneally. The right jugular vein and femoral artery were cannulated aseptically. The jugular vein was used for withdrawing blood and for resuscitation. The arterial catheter was used for continuous assessment of mean arterial pressure (MAP). The animal's body temperature was kept at 37° C. with a heating pad. T/HS was initiated by a 4-cm midline incision closed in two layers, followed by blood withdrawal from the venous catheter into a syringe containing 100 units of heparin sodium (Baxter Healthcare Corporation, Deerfield, Ill.). The MAP was reduced to 40 mm Hg in 10-15 minutes and maintained at 30-40 mm Hg for 90 minutes by further withdrawal or infusion of shed blood. At the end of the shock period animals were resuscitated by return of the shed blood. Animals were sacrificed under anesthesia by cardiac puncture and exsanguination at 3 hours and at 1, 3, and 7 days. Rat plasma was collected at each time point. The liver was harvested and flash frozen at −80° C. until the time of study.

Rat Intravenous Administration of MTD

Rats were treated with systemic MTD delivered by tail vein injection. Rats were placed in a restrainer and the tail was dipped in warm water for 1 minute. The tail vein was then punctured atraumatically with a 25 gauge needle. Rat liver-MTD (equivalent to MTD from 1% of the rat's liver as estimated by weight) was injected (Hauser et al., Shock 24(Suppl. 1):24-32, 2005). This yields a predicted plasma mtDNA concentration of 15 μg/mL. Rats were sacrificed 1 hour after injection by cardiac puncture and exsanguination. Rat liver and plasma were harvested, snap-frozen, and stored at −80° C. until use. Liver tissue samples were later thawed, weighed, and homogenized in T-PER reagent (Pierce, Rockford, Ill.) using a ratio of 1 g of tissue to 20 mL T-PER. Protein concentration was determined by BCA assay (Pierce, Rockford, Ill.).

Cytokine ELISA

TNF-α and IL-6 in rat liver were measured by BD OptEIA rat TNF ELISA set and rat IL-6 ELISA set (BD Biosciences, San Diego, Calif.), respectively, according to the manufacturer's protocol.

### Example 3

### Pancreatitis Releases Mitochondrial DAMPs into the Pancreatic Fluid

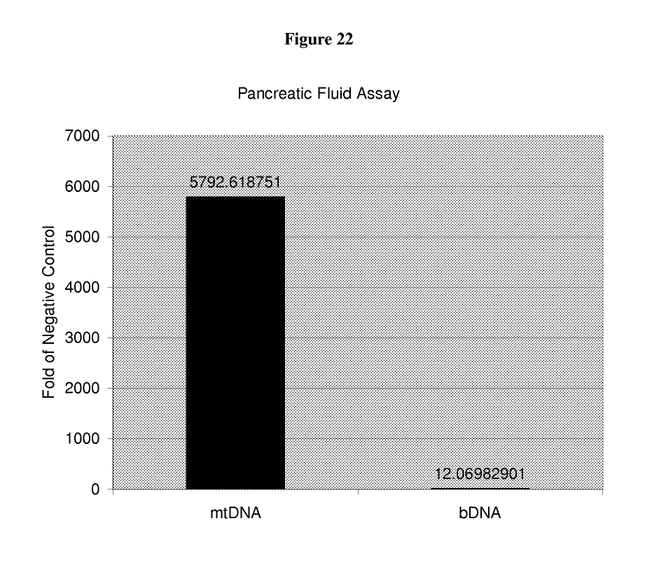

Additional experiments were performed to determine whether mtDNA is released into the pancreatic fluid in a rat having pancreatitis. In these experiments, the amount of mtDNA and bacterial DNA in pancreatic fluid in control rats and a rat model of pancreatitis were determined using real-time PCR. The fold-increase in mtDNA and bacterial DNA in pancreatic fluid relative to control is depicted. The resulting data show that mtDNA is increased in the pancreatic fluid in a rat having pancreatitis (FIG. 22). These data indicate that pancreatitis also releases mtDNA into the pancreatic fluid of a subject.

### Materials and Methods

DNA was isolated from 200 μL pancreatic juice and eluted with 80 μL water. Real-time qPCR was performed on the samples using primers for mitochondrial cytochrome B (mtDNA) and 16S rRNA (bDNA) as described above. Water was used as a negative control.

From the foregoing description, one skilled in the art can easily ascertain the essential characteristics of this invention; can make various changes and modifications of the invention to adapt it to various usages and conditions. Thus, other embodiments are also within the claims. All references cited herein are incorporated by reference in their entirety.

What is claimed is:

1. A method for predicting the likelihood that a subject will develop a sterile inflammation comprising the steps of:
a) measuring the amount of bacterial nucleic acid or peptide in a sample from said subject;
b) measuring the amount of a mitochondrial nucleic acid or peptide in said sample; and
c) comparing the amount of bacterial nucleic acid or peptide measured in step (a) with the amount of mitochondrial nucleic acid or peptide measured in step (b), wherein an increased ratio of the amount of mitochondrial nucleic acid or peptide to the amount of bacterial nucleic acid or peptide indicates a subject with an increased likelihood of later developing a sterile inflammation.

2. The method of claim 1, wherein said subject has experienced tissue damage.

3. The method of claim 2, wherein said tissue damage occurs as a result of surgery, hypotension, hypofusion/reperfusion injury, chemotherapy, pancreatitis, or shock.

4. The method of claim 2, wherein said tissue damage is not a result of blunt trauma.

5. The method of claim 1, wherein said sample is obtained from said subject within 2 hours of tissue damage.

6.-8. (canceled)

9. The method of claim 1, wherein said subject does not exhibit symptoms of systemic inflammation.

10. The method of claim 1, wherein said sterile inflammation is a systemic inflammation.

11. The method of claim 1, wherein the mitochondrial nucleic acid encodes cytochrome B, cytochrome C oxidase subunit III, or NADH dehydrogenase.

12. The method of claim 11, wherein the mitochondrial nucleic acid encodes cytochrome B.

13.-24. (canceled)

25. A method of determining whether a subject with tissue damage should be administered an antimicrobial agent or a reduced dosage of an antimicrobial agent, comprising the steps of:
a) measuring the amount of bacterial nucleic acid or peptide in a sample from said subject;
b) measuring the amount of a mitochondrial nucleic acid or peptide in said sample; and
c) comparing the amount of bacterial nucleic acid or peptide measured in step (a) with the amount of mitochondrial nucleic acid or peptide measured in step (b), wherein a subject having an increased ratio of the amount of mitochondrial nucleic acid or peptide to the amount of bacterial nucleic acid or peptide should not be administered an antimicrobial agent or should be administered a reduced dosage of an antimicrobial agent.

26. The method of claim 25, wherein said tissue damage occurs as a result of surgery, hypotension, hypofusion/reperfusion injury, chemotherapy, pancreatitis, or shock.

27. The method of claim 25, wherein said tissue damage is not a result of blunt trauma.

28. The method of claim 25, wherein said sample is obtained from said subject within 2 hours of tissue damage.

29.-31. (canceled)

32. The method of claim 25, wherein said subject does not exhibit symptoms of systemic inflammation.

33. The method of claim 25, wherein the mitochondrial nucleic acid encodes cytochrome B, cytochrome C oxidase subunit III, or NADH dehydrogenase.

34. The method of claim 33, wherein the mitochondrial nucleic acid encodes cytochrome B.

35. The method of claim 25 further comprising
d) administering to said subject having an increased ratio of the amount of mitochondrial nucleic acid or peptide to the amount of bacterial nucleic acid or peptide one or more anti-inflammatory agents and not administering, or administering at a reduced dosage, an antimicrobial agent.

36. The method of claim 35, wherein the one or more anti-inflammatory agents is selected from the group consisting of: cyclosporin H, anti-FPR antibodies, CpG Oligodeoxynucleotides, chloroquin, and anti-TLR9 antibodies.

37. The method of claim 35, wherein said tissue damage occurs as a result of surgery, hypotension, hypofusion/reperfusion injury, pancreatitis, or chemotherapy.

38.-45. (canceled)

46. A kit comprising:
a) a first reagent comprising one or more first oligonucleotide primers effective for the amplification of a bacterial nucleic acid or one or more antibodies that specifically bind to one or more bacterial peptides;
b) a second reagent comprising one or more second oligonucleotide primers effective for the amplification of a mitochondrial nucleic acid or one or more antibodies that specifically bind to one or more mitochondrial peptides; and
c) instructions for using said first and second reagents to identify a subject having an increased risk of of developing a sterile inflammation.

47. (canceled)

48. The kit of claim 46, whereby said instructions further inform a user how to determine whether the subject should be administered an antimicrobial agent or a reduced dosage of an antimicrobial agent if the subject has tissue damage.

49. The kit of claim 46, wherein the mitochondrial nucleic acid encodes cytochrome B, cytochrome C oxidase subunit III, or NADH dehydrogenase.

50. The kit of claim 49, wherein the mitochondrial nucleic acid encodes cytochrome B.

51.-55. (canceled)
